(19) United States
(12) Patent Application Publication
(10) Pub. No.: US 2022/0213557 A1
(43) Pub. Date: Jul. 7, 2022

(54) NON-CODING RNA FOR SUBTYPING OF BLADDER CANCER

(71) Applicants: DECIPHER BIOSCIENCES, INC., San Diego, CA (US); Erasmus University Medical Center, Rotterdam (NL)

(72) Inventors: Joep Jacobus de Jong, Rotterdam (NL); Yang Liu, Vancouver (CA); Ewan A. Gibb, Vancouver (CA); Elai Davicioni, San Diego, CA (US)

(21) Appl. No.: 17/611,545

(22) PCT Filed: May 12, 2020
(86) PCT No.: PCT/US2020/032484

§ 371 Date: Nov. 15, 2021

Related U.S. Application Data
(60) Provisional application No. 62/848,799, filed on May 16, 2019.

(51) Int. Cl. C12Q 1/6886

(52) U.S. Cl. CPC C12Q 1/6886; C12Q 2600/106; C12Q 2600/178; C12Q 2600/118; C12Q 2600/158; C12Q 2600/112

(57) ABSTRACT

The present disclosure pertains to the field of personalized medicine and methods for treating bladder cancer. In some embodiments, the disclosure relates to the use of long non-coding RNA (lncRNA) and genomic signatures for the prognosis of individuals with bladder cancer. The present disclosure provides methods for subtyping bladder cancer. The present disclosure also provides methods and compositions for treating bladder cancer.

### CROSS-REFERENCE TO RELATED APPLICATIONS

This application claims priority to U.S. Ser. No. 62/848,799, filed May 16, 2019, the entire contents of which is incorporated herein by reference in its entirety.

### FIELD

The present disclosure pertains to the field of personalized medicine and methods for treating bladder cancer. In particular, embodiments of the disclosure relate to the use of long non-coding RNA (lncRNA) and genomic signatures for the subtyping and/or prognosis of individuals with bladder cancer. Embodiments of the present disclosure provide methods for subtyping bladder cancer. Embodiments of the present disclosure also provide methods and compositions for treating bladder cancer.

### BACKGROUND

Bladder cancer has a global annual incidence of 430,000 patients, making it the fourth and tenth most common malignancy in men and women, respectively (Torre L A et al. (2012) CA Cancer J Clin. 2015; 65:87-108). Approximately 25% of patients present with muscle-invasive bladder cancer (MIBC). The recommended treatment option for MIBC is neoadjuvant cisplatin-based chemotherapy (NAC) followed by pelvic lymph node dissection and radical cystectomy (RC) (Grossman H B et al. (2003) N Engl J Med. 349:859-66 and International Collaboration of T et al. (2011) J Clin Oncol. 29:2171-7). Despite this aggressive treatment regimen, the 5-year overall survival (OS) is only approximately 55% from the time of surgery.

### SUMMARY

In recent years, gene expression profiling has revealed that MIBC is a heterogeneous disease; like breast cancer, it can be stratified into different molecular subtypes (Damrauer J S et al. (2014) Proc Natl Acad Sci. 111:3110-5; Sjodahl G et al. (2012) Clin Cancer Res. 18:3377-86; McConkey D J et al. (2018) Curr Oncol Rep. 2018; 20:77; and Cancer Genome Atlas Research N. (2014) Nature 507:315-22). At the highest level, there is a division into basal and luminal subtypes, with different models providing additional subdivisions (Choi W et al. (2014) Nat Rev Urol. 11:400-10 and Sjodahl G et al. (2017) J Pathol 242:113-25). Stratifying MIBC by molecular subtype has potential clinical value in terms of predicting both outcome and response to treatment, such as NAC or immunotherapy (Choi W et al. (2014) Cancer Cell 25:152-65; Seiler R et al. (2017) Eur Urol 72:544-54; and Robertson A G et al. (2017) Cell 171:540-56 e25).

While most MIBC studies to date have exclusively used messenger RNA (mRNA) expression to differentiate molecular subtypes, the mammalian transcriptome is comprised of a diverse range of coding (mRNA) and non-coding RNAs. Long non-coding RNAs (lncRNAs) are mRNA-like transcripts that range in length from 200 nucleotides to over 100 kilobases and lack open reading frames (Gutschner T et al. (2012) RNA Biol 9:703-19). They represent a significant fraction of the transcriptome, and, while it is unclear how many lncRNAs have biological function, their expression patterns can be specific to a particular biological or disease state (Gibb E A et al. (2011) PLoS One 6:e25915; Nguyen Q et al. (2016) Curr Top Microbiol Immunol 394:237-58). In The Cancer Genome Atlas (TCGA) study the lncRNA transcriptome divided the luminal-papillary subtype into two groups with distinct prognosis (Robertson A G et al. (2017) Cell 171:540-56 e25). There remains a need in the art for improved methods for subtyping bladder cancer to identify subjects with favorable prognosis.

The present disclosure relates to methods, compositions, systems and kits for the diagnosis, prognosis, and treatment of bladder cancer in a subject. The disclosure also provides biomarkers that define subgroups of bladder cancer, clinically useful classifiers for distinguishing bladder cancer subtypes, bioinformatic methods for determining clinically useful classifiers, and methods of use of each of the foregoing. The methods, compositions, systems and kits can provide expression-based analysis of biomarkers for purposes of subtyping bladder cancer in a subject. Further disclosed herein, in certain instances, are probe sets for use in subtyping bladder cancer in a subject. Classifiers for subtyping a bladder cancer and methods of treating bladder cancer based on molecular subtyping are also provided.

In one embodiment, the present disclosure provides a method comprising: a) providing a biological sample from a subject having bladder cancer; b) detecting the presence or expression level in the biological sample for a plurality of targets selected from Table 2 and/or Table 5; and c) subtyping the bladder cancer in the subject according to a genomic subtyping classifier based on the presence or expression levels of the plurality of targets, wherein said subtyping comprises assigning the bladder cancer to one of five subtypes selected from the group consisting of basal/squamous, luminal, luminal-infiltrated, luminal-papillary, and neuronal subtype. In some embodiments, the method further comprises determining that the subject has a favorable prognosis if the subtyping indicates that the subject has the luminal-papillary subtype or determining that the subject has an unfavorable prognosis if the subtyping indicates that the subject has the basal/squamous, luminal, luminal-infiltrated, or neuronal subtype. In some embodiments, the bladder cancer is FGFR3 positive. In some embodiments, the method further comprises determining that the subject has a less aggressive tumor if the subtyping indicates that the subject has the luminal-papillary subtype or determining that the subject has a more aggressive tumor if the subtyping indicates that the subject has the basal/squamous, luminal, luminal-infiltrated, or neuronal subtype. In still other embodiments, the methods further comprise administering an FGFR3 inhibitor to the subject if the subtyping indicates that the subject has the luminal-papillary subtype and administering neoadjuvant chemotherapy to the subject if the subtyping indicates that the subject has the basal/squamous, luminal, luminal-infiltrated, or neuronal subtype.

The disclosure provides a method for treating a subject with bladder cancer, the method comprising: determining the subtype of bladder cancer the subject has by: obtaining or having obtained a biological sample from the subject; measuring or having measured the levels of expression in the biological sample of a plurality of genes selected from Table 2 and/or Table 5; and assigning the bladder cancer to one of five subtypes selected from the group consisting of basal/squamous, luminal, luminal-infiltrated, luminal-papillary, and neuronal subtype based on the levels of expression of the plurality of genes; and if the subject has luminal-papillary subtype, then administering an FGFR3 inhibitor, and if the subject has basal/squamous, luminal, luminal-infiltrated, or neuronal subtype, then administering to the subject neoadjuvant chemotherapy.

In some embodiments, the neoadjuvant chemotherapy comprises administering cisplatin. In some embodiments, the methods of the present disclosure are performed prior to treatment of the patient with anti-cancer therapy. In some embodiments, the subject is undergoing anti-cancer therapy. In some embodiments, the subject has muscle-invasive bladder cancer. In some embodiments, the subject is a human being. In some embodiments, the level of expression is increased or reduced compared to a control.

In some embodiments, the biological sample in the methods of the present disclosure is a biopsy. In some embodiments, the biological sample is a urine sample, a blood sample, or a bladder tumor sample. In some embodiments, the biological sample is a transurethral resection (TUR) specimen.

In some embodiment, the present disclosure provides a method for treating a subject with bladder cancer, the method comprising: a) providing a biological sample from the subject; b) detecting the presence or expression level in the biological sample for a plurality of targets selected from Table 2 and/or Table 5; and c) administering a treatment to the subject, wherein the treatment is selected from the group consisting of neoadjuvant chemotherapy or an anti-cancer treatment. In some embodiments, the anti-cancer treatment is selected from the group consisting of surgery, chemotherapy, radiation therapy, immunotherapy, biological therapy, hormonal therapy, and photodynamic therapy.

In some embodiments, the present disclosure provides a method for treating a subject with bladder cancer, the method comprising: a) providing a biological sample from the subject; b) detecting the presence or expression level in the biological sample for a plurality of targets selected from Table 2 and/or Table 5; c) subtyping the bladder cancer in the subject according to a genomic subtyping classifier based on the presence or expression levels of the plurality of targets, wherein said subtyping comprises assigning the bladder cancer to one of five subtypes selected from the group consisting of basal/squamous, luminal, luminal-infiltrated, luminal-papillary, and neuronal subtype; and d) administering neoadjuvant chemotherapy to the subject if the subtyping indicates that the subject has the basal/squamous, luminal, luminal-infiltrated, or neuronal subtype and administering an anti-cancer treatment other than the neoadjuvant chemotherapy to the subject if the subtyping indicates that the subject has the luminal-papillary subtype, wherein the anti-cancer treatment other than neoadjuvant chemotherapy is selected from the group consisting of an FGFR3-inhibitor, surgery, chemotherapy, radiation therapy, immunotherapy, biological therapy, hormonal therapy, and photodynamic therapy.

The present disclosure provides methods for subtyping bladder cancer. In some embodiments, the methods comprise detecting the presence or expression level in the biological sample for a plurality of targets selected from Table 2 and/or Table 5. In certain embodiments, the plurality of targets are selected from the group consisting of AC017060.1, AC019117.1, AC019117.2, ACOX1, ADAM10, ADIRF, AEBP1, AF038458.3, AGR2, AQP3, ATP8B1, BCAS1, BEX4, BHLHE41, BMP5, BTG2, CAT, CHI3L1, COLIA1, COL1A2, COL3A1, COL6A1, COL6A3, CTD-2340E1.2, CTD-2626G11.2, DHRS2, FGFR3, FRY, GPX2, GREMI, HMGCS2, HPGD, MECOM, PDE10A, PGAP1, PHGR1, PLCD3, POFIB, PPDPF, PTPN13, RNF128, RP11-172F10.1, RP11-473M20.16, RP11-488L18.10, RP11-58K22.1, RP11-706O15.3, SEMA5A, SEMA6A, SFRP4, SHH, SLC14A1, SLITRK6, SNURF, SORL1, SULF1, TBX3, TMPRSS4, TOX3, UGT1A1, UGT1A3, UGT1A5, UGT1A8, UGT1A9, ZNF626, and ZNF737. In some embodiments, the plurality of targets are selected from the group consisting of FGFR3, TP53, RB1, CDKN1A, KMT2A, SSH3, STAG2, SF3B1, ERCC2, KDM6A, TSC1, NFE2L2, KRAS, SPTAN1, HRAS, ARID1A, HIST1H3B, PTEN, NRAS, NUP93, ACTB, RXRA, PARD3, EP300, ZNF773, MB21D2, TAF11, RHOA, ASXL1, KLF5, CDKN2A, TMCO4, KMT2D, MBD1, METTL3, PIK3CA, CULI, PSIPI, ERBB3, CREBBP, HESI, SF1, GNA13, KMT2C, KANSLI, ZFP36L1, ERBB2, ASXL2, ATM, C3orf70, CASP8, ELF3, FATI, FBXW7, RBM10, RHOB, SPN, USP28, and ZBTB7B.

The disclosure provides a method for subtyping and treating a subject for bladder cancer, the method comprising: a) providing a biological sample from the subject; b) detecting the presence or expression level in the biological sample for a plurality of targets selected from Table 2 and/or Table 5; and c) subtyping the bladder cancer in the subject according to a genomic subtyping classifier based on the presence or expression levels of the plurality of targets, wherein said subtyping comprises assigning the bladder cancer to one of five subtypes selected from the group consisting of basal/squamous, luminal, luminal-infiltrated, luminal-papillary, and neuronal subtype; and d) administering neoadjuvant chemotherapy to the subject if the subtyping indicates that the subject has the basal/squamous, luminal, luminal-infiltrated, or neuronal subtype and administering an anti-cancer treatment other than the neoadjuvant chemotherapy to the subject if the subtyping indicates that the subject has the luminal-papillary subtype, wherein the anti-cancer treatment other than neoadjuvant chemotherapy is selected from the group consisting of an FGFR3 inhibitor, surgery, chemotherapy, radiation therapy, immunotherapy, biological therapy, hormonal therapy, and photodynamic therapy. In one embodiment, the neoadjuvant chemotherapy comprises administering cisplatin. In some embodiment, the subject has muscle-invasive bladder cancer. In some embodiments, the bladder cancer is FGFR3 positive. In some embodiments, the biological sample comprises bladder cancer cells. In some embodiments, the biological sample comprises nucleic acids (e.g., RNA or DNA).

The method may be performed prior to treatment of the subject with neoadjuvant chemotherapy to determine if the subject will benefit from neoadjuvant chemotherapy or should be administered some other anti-cancer treatment. The method may also be performed while the subject is undergoing neoadjuvant chemotherapy to help evaluate whether continued treatment is likely to be efficacious. In some embodiments, the subject is treated with an FGFR3 inhibitor.

The biological sample obtained from a patient is typically urine, a biopsy, blood or a tumor sample, but can be any sample from bodily fluids or tissue of the patient that contains cancerous cells or nucleic acids (e.g., RNA, DNA). In certain embodiments, nucleic acids (e.g., RNA transcripts) comprising sequences from targets selected from Table 2 and/or Table 5, or complements thereof, are further isolated from the biological sample, and/or purified, and/or amplified prior to analysis.

The presence or expression level of biomarker nucleic acids can be determined by using a variety of methods including, but not limited to, in situ hybridization, a PCR-based method, an array-based method, an immunohistochemical method, a sequencing method (e.g., next-generation sequencing), an RNA assay method, or an immunoassay method. In some embodiments, the level of expression is determined using a reagent selected from the group consisting of a nucleic acid probe, one or more nucleic acid primers, and an antibody. In certain embodiments, the presence or expression level of biomarker nucleic acids can be determined by measuring the level of an RNA transcript. In some embodiments, the RNA is long non-coding RNA.

In some embodiments, the disclosure provides a method of subtyping bladder cancer in a subject, comprising: providing a biological sample from the subject, and detecting the presence or expression level in the biological sample for a plurality of targets selected from Table 2 and/or Table 5; wherein the presence or expression level of the plurality of targets provides an indication of the bladder cancer subtype. In some embodiments, the biological sample comprises bladder cancer cells. In some embodiments, the biological sample comprises nucleic acids. In some embodiments, the expression level of at least one target is reduced compared to a control. In some embodiments, the expression level of at least one target is increased compared to a control. In yet other embodiments, the level of expression of at least one target is determined by using a method selected from the group consisting of in situ hybridization, a PCR-based method, an array-based method, an immunohistochemical method, a sequencing method, an RNA assay method and an immunoassay method. In some embodiments, the level of expression of at least one target is detected using a reagent selected from the group consisting of a nucleic acid probe, one or more nucleic acid primers, and an antibody. In still other embodiments, determining the level of expression comprises measuring the level of a nucleic acid (e.g., RNA transcript).

The disclosure provides a method for determining a treatment for a subject who has bladder cancer, the method comprising: a) providing a biological sample from the subject; b) detecting the presence or expression level in the biological sample for a plurality of targets selected from Table 2 and/or Table 5; c) subtyping the bladder cancer of the subject according to a genomic subtyping classifier based on the presence or expression levels of the plurality of targets, wherein said subtyping comprises assigning the bladder cancer to one of five subtypes selected from the group consisting of basal/squamous, luminal, luminal-infiltrated, luminal-papillary, and neuronal subtype; and d) determining whether or not the subject is likely to be responsive to anti-cancer therapy based on the subtype of the bladder cancer in the subject; and e) prescribing anti-cancer therapy to the subject if the patient is identified as likely to be responsive to anti-cancer therapy. In some embodiments, the bladder cancer is FGFR3 positive. In some embodiments, the biological sample comprises bladder cancer cells. In some embodiments, the biological sample comprises nucleic acids. In some embodiments, the anti-cancer therapy is an FGFR3 inhibitor.

The disclosure provides a probe set for prognosing bladder cancer in a subject, the probe set comprising a plurality of probes for detecting a plurality of targets, wherein the plurality of targets comprises one or more target sequences, or complements thereof, of targets selected from Table 2 and/or Table 5, or a combination thereof. Probes may be detectably labeled to facilitate detection. In some embodiments, the bladder cancer is FGFR3 positive.

The disclosure provides a system for analyzing a bladder cancer to provide a prognosis to a subject having bladder cancer, the system comprising: a) a probe set described herein; and b) a computer model or algorithm for analyzing an expression level or expression profile of the plurality of target nucleic acids hybridized to the plurality of probes in a biological sample from a subject who has bladder cancer and subtyping the bladder cancer of the subject according to a genomic subtyping classifier based on the expression level or expression profile, wherein said subtyping comprises assigning the bladder cancer to one of five subtypes selected from the group consisting of basal/squamous, luminal, luminal-infiltrated, luminal-papillary, and neuronal subtype. In some embodiments, the bladder cancer is FGFR3 positive.

The disclosure provides a kit for prognosing bladder cancer in a subject, the kit comprising agents for detecting the presence or expression levels for a plurality of targets, wherein said plurality of targets comprises one or more targets selected from Table 2 and/or Table 5. In some embodiments, the bladder cancer is FGFR3 positive. The kit may include one or more agents (e.g., hybridization probes, PCR primers, or microarray) for detecting the presence or expression levels of a plurality of targets, wherein said plurality of targets comprises one or more targets selected from Table 2 and/or Table 5, a container for holding a biological sample isolated from a human subject for testing, and printed instructions for reacting the agents with the biological sample or a portion of the biological sample to determine whether or not the subject is likely to benefit from neoadjuvant chemotherapy. In some embodiments, the biological sample comprises bladder cancer cells. In some embodiments, the biological sample comprises nucleic acids (e.g., RNA or DNA). The agents may be packaged in separate containers. The kit may further comprise one or more control reference samples or other reagents for measuring gene expression (e.g., reagents for performing PCR, RT-PCR, microarray analysis, a Northern blot, an immunoassay, or immunohistochemistry). In one embodiment, the kit comprises agents for detecting the presence or expression levels of the targets listed in Table 2 and/or Table 5. In some embodiment, the kit comprises agents for detecting the presence or expression levels of all the targets listed in Table 2 and/or Table 5. For example, the kit may comprise a probe set, as described herein, for detecting a plurality of targets, wherein the plurality of targets comprises one or more target sequences, or complements thereof, of targets selected from Table 2 and/or Table 5, or any combination thereof.

In some embodiment, the kit further comprises a system for analyzing a bladder cancer to predict response of a subject to neoadjuvant chemotherapy, wherein the system comprises: a) a probe set comprising a plurality of probes for detecting a plurality of target nucleic acids, wherein the plurality of target nucleic acids comprises one or more target sequences, or complements thereof, of targets selected from Table 2 and/or Table 5, or any combination thereof; and b) a computer model or algorithm for analyzing an expression level or expression profile of the plurality of target nucleic acids hybridized to the plurality of probes in a biological sample from a subject who has bladder cancer and subtyping the bladder cancer of the subject according to a genomic subtyping classifier based on the expression level or expression profile, wherein said subtyping comprises assigning the bladder cancer to one of five subtypes selected from the group consisting of basal/squamous, luminal, luminal-infiltrated, luminal-papillary, and neuronal subtype. In some embodiments, the bladder cancer is FGFR3 positive.

The disclosure provides a method of diagnosing, prognosing, determining the progression of cancer, or predicting benefit from therapy in a subject with bladder cancer, comprising: a) providing a biological sample from the subject; b) assaying an expression level in the biological sample from the subject for a plurality of targets using at least one reagent that specifically binds to said targets, wherein the plurality of targets comprises one or more targets selected from Table 2 and/or Table 5; and c) diagnosing, prognosing, determining the progression of cancer, or predicting benefit from therapy in the subject based on the expression level of the plurality of targets. In some embodiments, the biological sample comprises bladder cancer cells. In some embodiments, the biological sample comprises nucleic acids (e.g., RNA or DNA). In some embodiments, the bladder cancer is FGFR3 positive.

The significance of the expression levels of one or more biomarker targets of the present disclosure may be evaluated using, for example, a T-test, P-value, KS (Kolmogorov Smirnov) P-value, accuracy, accuracy P-value, positive predictive value (PPV), negative predictive value (NPV), sensitivity, specificity, AUC, AUC P-value (Auc.pvalue), Wilcoxon Test P-value, Median Fold Difference (MFD), Kaplan Meier (KM) curves, survival AUC (survAUC), Kaplan Meier P-value (KM P-value), Univariable Analysis Odds Ratio P-value (uvaORPval), multivariable analysis Odds Ratio P-value (mvaORPval), Univariable Analysis Hazard Ratio P-value (uvaHRPval) and Multivariable Analysis Hazard Ratio P-value (mvaHRPval). The significance of the expression level of the one or more targets may be based on two or more metrics selected from the group comprising AUC, AUC P-value (Auc.pvalue), Wilcoxon Test P-value, Median Fold Difference (MFD), Kaplan Meier (KM) curves, survival AUC (survAUC), Univariable Analysis Odds Ratio P-value (uvaORPval), multivariable analysis Odds Ratio P-value (mvaORPval), Kaplan Meier P-value (KM P-value), Univariable Analysis Hazard Ratio P-value (uvaHRPval) or Multivariable Analysis Hazard Ratio P-value (mvaHRPval).

These and other embodiments of the subject disclosure will readily occur to those of skill in the art in view of the disclosure herein.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

FIGS. 1A-1F show embodiments of consensus cluster plus output from unsupervised clustering of 750 highly variant lncRNAs in the NAC cohort. FIG. 1A shows 3-cluster solution,

FIG. 1B shows 4-cluster solution, FIG. 1C shows 5-cluster solution, FIG. 1D shows 6-cluster solution, FIG. 1E shows tracking plot, and FIG. 1F shows consensus Cumulative Distribution Function (CDF) plot.

FIGS. 2A-2F show embodiments of survival analysis for the lncRNA-based clustering solution. For the NAC cohort: FIG. 2A shows KM plots for lncRNA clusters (LC1-4) (survival at month 60: LC3>LC1>LC2>LC4), FIG. 2B shows Intersection of the lncRNA clusters (LC1-4) with the luminal papillary mRNA subtype, and FIG. 2C shows KM plots for lncRNA-split luminal papillary tumors (LPL-C3, LPL-Other) (survival at month 60: LPL-C3>RestSubtypes>LPL-Others). For the TCGA cohort: FIG. 2D shows KM plots for lncRNA clusters (LC1-4) (survival at month 60: LC1>LC3>LC2=LC4), FIG. 2E shows Intersection of the lncRNA clusters (LC1-4) with the luminal papillary mRNA subtype, and FIG. 2F shows KM plots for lncRNA-split luminal papillary tumors (LPL-C3, LPL-Other) (survival at month 60: LPL-C3>LPL-Others>RestSubtypes). For KM plots, the values in brackets indicate number of patients and events for each class.

FIGS. 3A-3B show embodiments of molecular subtyping of the NAC cohort using the TCGA 2017 classifier. FIG. 3A shows Heatmap of the five TCGA subtypes (luminal-papillary, luminal, luminal infiltrated, basal squamous and neuronal) and unknown. FIG. 3B shows Survival analysis of the NAC cohort stratified by the TCGA 2017 classifier (survival at month 60: Unknown>LumP>Ba/Sq; Luminf ends at ˜44 months; Neuronal ends at ˜16 months; Lum ends at ˜12 months).

FIGS. 4A-4B show embodiments of survival analysis for lncRNA clusters stratified by (A) luminal-papillary (survival at month 60: LC3>LC1>LC2>LC4) or (B) basal/squamous (survival at month 60: LC4>LC2>LC3>LC1) mRNA subtypes in the NAC cohort.

FIGS. 5A-5B show embodiments of biological characterization of the lncRNA clusters using selected MIBC marker genes in the (A) NAC and (B) TCGA cohorts (columns left to right are LC1-LC4, respectively). For the NAC and TCGA cohorts, both the five TCGA subtypes (luminal-papillary, luminal, luminal infiltrated, basal squamous and neuronal, unknown), and the luminal papillary subgroups (LPL-C3, LPL-Other and RestSubtypes) are indicated in the covariate tracks. In the TCGA cohort, the 2017 TCGA four-cluster lncRNA solution, FGFR3, TP53 and RB1 mutation status, and FGFR3 fusion status, are also indicated in covariate tracks.

FIGS. 6A-6F show embodiments of expression of select MIBC marker genes associated with the luminal subtype in the LPL-C3 and LPL-Other tumors, for the NAC cohort (A) PPARG, (B) FOXA1, (C) GATA3 and TCGA cohort (D) PPARG, (E) FOXA1, (F) GATA3, respectively.

FIGS. 7A-7D show embodiments of expression of select MIBC marker genes associated with the basal subtype in the LPL-C3 and LPL-Other tumors, for the NAC cohort (A) KRT5, (B) KRT14 and TCGA cohort (C) KRT5, (D) KRT14, respectively.

FIGS. 8A-8D show embodiments of expression of select MIBC marker genes associated with the immune oncology in the LPL-C3 and LPL-Other tumors, for the NAC cohort (A) CD274 (PD-L1), (B) PDCD1LG2 (PD-1) and TCGA cohort (C) CD274 (PD-L1), (D) PDCDILG2 (PD-1), respectively.

FIGS. 9A-9F show embodiments of expression of select genes associated with EMT in the LPL-C3 and LPL-Other tumors, for the NAC cohort (A) VIM, (B) ZEB1, (C) CDH1 and TCGA cohort (D) VIM, (E) ZEB1, (F) CDH1, respectively.

FIGS. 10A-10H show embodiments of biological pathways differentially regulated between LPL-C3 and LPL-Other tumors. For the NAC cohort, FIG. 10A shows EMT hallmark activity, FIG. 10B shows SHH-BMP pathway activity, FIG. 10C shows FGFR3 signature score, FIG. 10D shows p53 hallmark activity. The TCGA cohort follows the same order for panels E-H.

FIGS. 11A-11B show embodiments of sample purity estimates for the (A) NAC cohort using the ESTIMATE algorithm and (B) TCGA using the ABSOLUTE algorithm (previously calculated).

FIGS. 12A-12F show embodiments of expression of select genes associated with SHH and urothelial differentiation in the LPL-C3 and LPL-Other tumors, for the NAC cohort (A) SHH, (B) UPK3A, (C) UPK3B and TCGA cohort (D) SHH, (E) UPK3A, (F) UPK3B, respectively.

FIGS. 13A-13C show embodiments of regulon activities of the lncRNA-based consensus clusters. FIG. 13A shows mean regulon activities in lncRNA clusters for 16 regulators in the TCGA and NAC cohorts (columns left to right are LC1-LC4, respectively). Asterisks mark clusters that were significantly enriched (Fisher's Exact test, Benjamini Hochberg adjusted, p<10-3) with activated or repressed samples for a regulon. Regulons activities in the TCGA cohort for (B) SHH and (C) FGFR3, with TP53, FGFR3 and RB1 mutation status and LPL-C3 vs. LPL-Other indicated in covariate tracks. A dark black bar indicates a mutation event.

FIGS. 14A-14D show embodiments of correlation of gene expression and pathway activity with respect to mutation status in the TCGA cohort. FIG. 14A shows FGFR3 gene expression, FIG. 14B shows FGFR3 pathway activity, FIG. 14C shows TP53 gene expression and FIG. 14D shows p53 pathway activity. For each panel, ‘no’ indicates wild-type and ‘yes’ indicates a mutation.

FIGS. 15A-15C show embodiments of RB1 expression in (A) TCGA and (B) NAC cohorts. FIG. 15C shows RB1 regulon activity scores in TCGA cohort with TP53, FGFR3 and RB1 mutation status and LPL-C3 vs. LPL-Other indicated in covariate tracks. A dark black bar indicates a mutation event.

FIGS. 16A-16C show embodiments of survival analysis of FGFR3+ cases determined by the GC in three cohorts. (A) NAC (n=223) (survival at month 60: FGFR3+>RestSubtypes>Remaining LPs), (B) TCGA (n=405) (survival at month 60: FGFR3+>RestSubtypes>Remaining LPs>RestSubtypes) and (C) UTSW (n=94) (survival at month 60: FGFR3+>RestSubtypes>Remaining LPs).

FIGS. 17A-17H show embodiments of biological pathways differentially activated between tumors classed as FGFR3+ by the GC and other tumors. For the NAC cohort, FIG. 17A shows EMT hallmark activity, FIG. 17B shows SHH-BMP pathway activity, FIG. 17C shows FGFR3 signature score, and FIG. 17D shows p53 hallmark activity. The TCGA cohort follows the same order for panels E-H.

FIGS. 18A-18H show embodiments of biological pathways differentially active between tumors classed as FGFR3+ by the GC and other tumors. For the UTSW cohort, FIG. 18A shows EMT hallmark activity, FIG. 18B shows SHH-BMP pathway activity, FIG. 18C shows FGFR3 signature score, and FIG. 18D shows p53 hallmark activity. The PCC cohort follows the same order for panels E-H.

### DETAILED DESCRIPTION

The present disclosure discloses systems and methods for prognosing, subtyping, diagnosing, predicting, and/or monitoring the status or outcome of bladder cancer in a subject using expression-based analysis of a plurality of targets. Generally, the method comprises (a) optionally providing a sample from a subject; (b) assaying the expression level of a plurality of targets in the sample; and (c) prognosing, subtyping, diagnosing, predicting and/or monitoring the status or outcome of a bladder cancer based on the expression level of the plurality of targets. Assaying the expression level for a plurality of targets in the sample may comprise applying the sample to a microarray. In some instances, assaying the expression level may comprise the use of an algorithm. The algorithm may be used to produce a classifier. In some instances, the classifier may provide a probe selection region. In some instances, assaying the expression level for a plurality of targets comprises detecting and/or quantifying the plurality of targets. In some embodiments, assaying the expression level for a plurality of targets comprises sequencing the plurality of targets. In some embodiments, assaying the expression level for a plurality of targets comprises amplifying the plurality of targets. In some embodiments, assaying the expression level for a plurality of targets comprises quantifying the plurality of targets. In some embodiments, assaying the expression level for a plurality of targets comprises conducting a multiplexed reaction on the plurality of targets. In some instances, the plurality of targets comprises one or more targets selected from Table 2 and/or Table 5. In some instances, the plurality of targets comprises at least about 2, at least about 3, at least about 4, at least about 5, at least about 6, at least about 7, at least about 8, at least about 9, or at least about 10 targets selected from Table 2 and/or Table 5.

Further disclosed herein are methods for subtyping bladder cancer. Generally, the method comprises: (a) providing a sample from a subject; (b) assaying the expression level for a plurality of targets in the sample; and (c) subtyping the bladder cancer based on the expression level of the plurality of targets. In some instances, the plurality of targets comprises one or more targets selected from Table 2 and/or Table 5. In some embodiments, the biological sample comprises bladder cancer cells. In some embodiments, the biological sample comprises nucleic acids (e.g., RNA or DNA). In some instances, the plurality of targets comprises at least about 2, at least about 3, at least about 4, at least about 5, at least about 6, at least about 7, at least about 8, at least about 9, or at least about 10 targets selected from Table 2 and/or Table 5. In some instances, subtyping the bladder cancer comprises determining whether the cancer would respond to an anti-cancer therapy. In some instances, subtyping the bladder cancer comprises identifying the cancer as non-responsive to an anti-cancer therapy. Optionally, subtyping the bladder cancer comprises identifying the cancer as responsive to an anti-cancer therapy.

Before the present disclosure is described in further detail, it is to be understood that this disclosure is not limited to the particular methodology, compositions, articles or machines described, as such methods, compositions, articles or machines can, of course, vary. It is also to be understood that the terminology used herein is for the purpose of describing particular embodiments only, and is not intended to limit the scope of the present disclosure.

### Targets

In some instances, assaying the expression level of a plurality of genes comprises detecting and/or quantifying a plurality of target analytes. In some embodiments, assaying the expression level of a plurality of genes comprises sequencing a plurality of target nucleic acids. In some embodiments, assaying the expression level of a plurality of biomarker genes comprises amplifying a plurality of target nucleic acids. In some embodiments, assaying the expression level of a plurality of biomarker genes comprises conducting a multiplexed reaction on a plurality of target analytes.

The methods disclosed herein often comprise assaying the expression level of a plurality of targets. The plurality of targets may comprise coding targets and/or non-coding targets of a protein-coding gene or a non-protein-coding gene. A protein-coding gene structure may comprise an exon and an intron. The exon may further comprise a coding sequence (CDS) and an untranslated region (UTR). The protein-coding gene may be transcribed to produce a pre-mRNA and the pre-mRNA may be processed to produce a mature mRNA. The mature mRNA may be translated to produce a protein.

A non-protein-coding gene structure may comprise an exon and intron. Usually, the exon region of a non-protein-coding gene primarily contains a UTR. The non-protein-coding gene may be transcribed to produce a pre-mRNA and the pre-mRNA may be processed to produce a non-coding RNA (ncRNA).

A coding target may comprise a coding sequence of an exon. A non-coding target may comprise a UTR sequence of an exon, intron sequence, intergenic sequence, promoter sequence, non-coding transcript, CDS antisense, intronic antisense, UTR antisense, or non-coding transcript antisense. A non-coding transcript may comprise a non-coding RNA (ncRNA).

In some instances, the plurality of targets comprises one or more targets selected from Table 2 and/or Table 5. In some instances, the plurality of targets comprises at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, or at least about 50 targets selected from Table 2 and/or Table 5.

In some instances, the plurality of targets comprises a coding target, non-coding target, or any combination thereof. In some instances, the coding target comprises an exonic sequence. In some instances, the non-coding target comprises a non-exonic or exonic sequence. In some instances, a non-coding target comprises a UTR sequence, an intronic sequence, antisense, or a non-coding RNA transcript. In some instances, a non-coding target comprises sequences which partially overlap with a UTR sequence or an intronic sequence. A non-coding target also includes non-exonic and/or exonic transcripts. Exonic sequences may comprise regions on a protein-coding gene, such as an exon, UTR, or a portion thereof. Non-exonic sequences may comprise regions on a protein-coding, non-protein-coding gene, or a portion thereof. For example, non-exonic sequences may comprise intronic regions, promoter regions, intergenic regions, a non-coding transcript, an exon anti-sense region, an intronic anti-sense region, UTR anti-sense region, non-coding transcript anti-sense region, or a portion thereof. In some instances, the plurality of targets comprises a non-coding RNA transcript.

The plurality of targets may comprise one or more targets selected from a classifier disclosed herein. The classifier may be generated from one or more models or algorithms. The one or more models or algorithms may be Naïve Bayes (NB), recursive Partitioning (Rpart), random forest (RF), support vector machine (SVM), k-nearest neighbor (KNN), high dimensional discriminate analysis (HDDA), or a combination thereof. The classifier may have an AUC of equal to or greater than 0.60. The classifier may have an AUC of equal to or greater than 0.61. The classifier may have an AUC of equal to or greater than 0.62. The classifier may have an AUC of equal to or greater than 0.63. The classifier may have an AUC of equal to or greater than 0.64. The classifier may have an AUC of equal to or greater than 0.65. The classifier may have an AUC of equal to or greater than 0.66. The classifier may have an AUC of equal to or greater than 0.67. The classifier may have an AUC of equal to or greater than 0.68. The classifier may have an AUC of equal to or greater than 0.69. The classifier may have an AUC of equal to or greater than 0.70. The classifier may have an AUC of equal to or greater than 0.75. The classifier may have an AUC of equal to or greater than 0.77. The classifier may have an AUC of equal to or greater than 0.78. The classifier may have an AUC of equal to or greater than 0.79. The classifier may have an AUC of equal to or greater than 0.80. The AUC may be clinically significant based on its 95% confidence interval (CI). The accuracy of the classifier may be at least about 70%. The accuracy of the classifier may be at least about 73%. The accuracy of the classifier may be at least about 75%. The accuracy of the classifier may be at least about 77%. The accuracy of the classifier may be at least about 80%. The accuracy of the classifier may be at least about 83%. The accuracy of the classifier may be at least about 84%. The accuracy of the classifier may be at least about 86%. The accuracy of the classifier may be at least about 88%. The accuracy of the classifier may be at least about 90%. The p-value of the classifier may be less than or equal to 0.05. The p-value of the classifier may be less than or equal to 0.04. The p-value of the classifier may be less than or equal to 0.03. The p-value of the classifier may be less than or equal to 0.02. The p-value of the classifier may be less than or equal to 0.01. The p-value of the classifier may be less than or equal to 0.008. The p-value of the classifier may be less than or equal to 0.006. The p-value of the classifier may be less than or equal to 0.004. The p-value of the classifier may be less than or equal to 0.002. The p-value of the classifier may be less than or equal to 0.001.

The plurality of targets may comprise one or more targets selected from a Random Forest (RF) classifier. The plurality of targets may comprise two or more targets selected from a Random Forest (RF) classifier. The plurality of targets may comprise three or more targets selected from a Random Forest (RF) classifier. The plurality of targets may comprise 2, 3, 4, 5, 6, 7, 8, 9, 10, 11, 12, 13, 14, 15, 20, 25, 30, 35, 40, 45, 50, or more targets selected from a Random Forest (RF) classifier, or a range defined by any two of the aforementioned numbers of targets. The RF classifier may be an RF2, and RF3, or an RF4 classifier. The RF classifier may be an RF22 classifier (e.g., a Random Forest classifier with 22 targets). For example, a RF classifier of the present disclosure may comprise two or more targets selected from Table 2 and/or Table 5.

The plurality of targets may comprise one or more targets selected from an SVM classifier. The plurality of targets may comprise 2, 3, 4, 5, 6, 7, 8, 9, 10 or more targets selected from an SVM classifier. The plurality of targets may comprise 12, 13, 14, 15, 17, 20, 22, 25, 27, 30 or more targets selected from an SVM classifier. The plurality of targets may comprise 32, 35, 37, 40, 43, 45, 47, 50, 53, 55, 57, 60 or more targets selected from an SVM classifier, or a range defined by any two of the aforementioned numbers of targets. The SVM classifier may be an SVM2 classifier. A SVM classifier of the present disclosure may comprise two or more targets selected from Table 2 and/or Table 5.

The plurality of targets may comprise one or more targets selected from a KNN classifier. The plurality of targets may comprise 2, 3, 4, 5, 6, 7, 8, 9, 10 or more targets selected from a KNN classifier. The plurality of targets may comprise 12, 13, 14, 15, 17, 20, 22, 25, 27, 30 or more targets selected from a KNN classifier. The plurality of targets may comprise 32, 35, 37, 40, 43, 45, 47, 50, 53, 55, 57, 60 or more targets selected from a KNN classifier. The plurality of targets may comprise 65, 70, 75, 80, 85, 90, 95, 100 or more targets selected from a KNN classifier, or a range defined by any two of the aforementioned numbers of targets. For example, a KNN classifier of the present disclosure may comprise two or more targets selected from Table 2 and/or Table 5.

The plurality of targets may comprise one or more targets selected from a Naïve Bayes (NB) classifier. The plurality of targets may comprise 2, 3, 4, 5, 6, 7, 8, 9, 10 or more targets selected from an NB classifier. The plurality of targets may comprise 12, 13, 14, 15, 17, 20, 22, 25, 27, 30 or more targets selected from an NB classifier. The plurality of targets may comprise 32, 35, 37, 40, 43, 45, 47, 50, 53, 55, 57, 60 or more targets selected from a NB classifier. The plurality of targets may comprise 65, 70, 75, 80, 85, 90, 95, 100 or more targets selected from a NB classifier, or a range defined by any two of the aforementioned numbers of targets. For example, a NB classifier of the present disclosure may comprise two or more targets selected from Table 2 and/or Table 5.

The plurality of targets may comprise one or more targets selected from a recursive partitioning (Rpart) classifier. The plurality of targets may comprise 2, 3, 4, 5, 6, 7, 8, 9, 10 or more targets selected from an Rpart classifier. The plurality of targets may comprise 12, 13, 14, 15, 17, 20, 22, 25, 27, 30 or more targets selected from an Rpart classifier. The plurality of targets may comprise 32, 35, 37, 40, 43, 45, 47, 50, 53, 55, 57, 60 or more targets selected from an Rpart classifier. The plurality of targets may comprise 65, 70, 75, 80, 85, 90, 95, 100 or more targets selected from an Rpart classifier, or a range defined by any two of the aforementioned numbers of targets. For example, an Rpart classifier of the present disclosure may comprise two or more targets selected from Table 2 and/or Table 5.

The plurality of targets may comprise one or more targets selected from a high dimensional discriminate analysis (HDDA) classifier. The plurality of targets may comprise two or more targets selected from a high dimensional discriminate analysis (HDDA) classifier. The plurality of targets may comprise three or more targets selected from a high dimensional discriminate analysis (HDDA) classifier. The plurality of targets may comprise 5, 6, 7, 8, 9, 10, 11, 12, 13, 14, 15 or more targets selected from a high dimensional discriminate analysis (HDDA) classifier, or a range defined by any two of the aforementioned numbers of targets. For example, an Rpart classifier of the present disclosure may comprise two or more targets selected from Table 2 and/or Table 5.

### Probes/Primers

The present disclosure provides a probe set for subtyping and/or prognosing, diagnosing, monitoring and/or predicting a status or outcome of bladder cancer in a subject, the probe set comprising a plurality of probes, wherein (i) the probes in the set are capable of detecting an expression level of at least one target selected from Table 2 and/or Table 5; and (ii) the expression level determines the cancer status of the subject with at least about 40% specificity.

The probe set may comprise one or more polynucleotide probes. Individual polynucleotide probes comprise a nucleotide sequence derived from the nucleotide sequence of the target sequences or complementary sequences thereof. The nucleotide sequence of the polynucleotide probe is designed such that it corresponds to, or is complementary to the target sequences. The polynucleotide probe can specifically hybridize under either stringent or lowered stringency hybridization conditions to a region of the target sequences, to the complement thereof, or to a nucleic acid sequence (such as a cDNA) derived therefrom.

The selection of the polynucleotide probe sequences and determination of their uniqueness may be carried out in silico using techniques known in the art, for example, based on a BLASTN search of the polynucleotide sequence in question against gene sequence databases, such as the Human Genome Sequence, UniGene, dbEST or the non-redundant database at NCBI. In one embodiment of the disclosure, the polynucleotide probe is complementary to a region of a target mRNA derived from a target sequence in the probe set. Computer programs can also be employed to select probe sequences that may not cross hybridize or may not hybridize non-specifically.

In some instances, microarray hybridization of RNA, extracted from bladder cancer tissue samples and amplified, may yield a dataset that is then summarized and normalized by the fRMA technique. After removal (or filtration) of cross-hybridizing PSRs, and PSRs containing less than 4 probes, the remaining PSRs can be used in further analysis. Following fRMA and filtration, the data can be decomposed into its principal components and an analysis of variance model is used to determine the extent to which a batch effect remains present in the first 10 principal components.

These remaining PSRs can then be subjected to filtration by a T-test between CR (clinical recurrence) and non-CR samples. Using a p-value cut-off of 0.01, the remaining features (e.g., PSRs) can be further refined. Feature selection can be performed by regularized logistic regression using the elastic-net penalty. The regularized regression may be bootstrapped over 1000 times using all training data; with each iteration of bootstrapping, features that have non-zero co-efficient following 3-fold cross validation can be tabulated. In some instances, features that were selected in at least 25% of the total runs were used for model building.

The polynucleotide probes of the present disclosure may range in length from about 15 nucleotides to the full length of the coding target or non-coding target. In one embodiment of the methods, systems and compositions, the polynucleotide probes are at least about 15 nucleotides in length. In some embodiment, the polynucleotide probes are at least about 20 nucleotides in length. In a further embodiment, the polynucleotide probes are at least about 25 nucleotides in length. In some embodiment, the polynucleotide probes are between about 15 nucleotides and about 500 nucleotides in length. In some embodiments, the polynucleotide probes are between about 15 nucleotides and about 450 nucleotides, about 15 nucleotides and about 400 nucleotides, about 15 nucleotides and about 350 nucleotides, about 15 nucleotides and about 300 nucleotides, about 15 nucleotides and about 250 nucleotides, about 15 nucleotides and about 200 nucleotides in length. In some embodiments, the probes are at least 15 nucleotides in length. In some embodiments, the probes are at least 15 nucleotides in length. In some embodiments, the probes are at least 20 nucleotides, at least 25 nucleotides, at least 50 nucleotides, at least 75 nucleotides, at least 100 nucleotides, at least 125 nucleotides, at least 150 nucleotides, at least 200 nucleotides, at least 225 nucleotides, at least 250 nucleotides, at least 275 nucleotides, at least 300 nucleotides, at least 325 nucleotides, at least 350 nucleotides, at least 375 nucleotides in length.

The polynucleotide probes of a probe set can comprise RNA, DNA, RNA or DNA mimetics, or combinations thereof, and can be single-stranded or double-stranded. Thus the polynucleotide probes can be composed of naturally-occurring nucleobases, sugars and covalent internucleoside (backbone) linkages as well as polynucleotide probes having non-naturally-occurring portions which function similarly. Such modified or substituted polynucleotide probes may provide desirable properties such as, for example, enhanced affinity for a target gene and increased stability. The probe set may comprise a coding target and/or a non-coding target. Preferably, the probe set comprises a combination of a coding target and non-coding target.

In some embodiments, the probe set comprise a plurality of target sequences that hybridize to at least about 5 coding targets and/or non-coding targets selected from Table 2 and/or Table 5. In some instances, the probe set comprise a plurality of target sequences that hybridize to at least about 10 coding targets and/or non-coding targets selected from Table 2 and/or Table 5. In some embodiments, the probe set comprise a plurality of target sequences that hybridize to at least about 15 coding targets and/or non-coding targets selected from Table 2 and/or Table 5. In some embodiments, the probe set comprise a plurality of target sequences that hybridize to at least about 20 coding targets and/or non-coding targets selected from Table 2 and/or Table 5. In some embodiments, the probe set comprise a plurality of target sequences that hybridize to at least about 30 coding targets and/or non-coding targets selected from Table 2 and/or Table 5.

The system of the present disclosure further provides for primers and primer pairs capable of amplifying target sequences defined by the probe set, or fragments or subsequences or complements thereof. The nucleotide sequences of the probe set may be provided in computer-readable media for in silico applications and as a basis for the design of appropriate primers for amplification of one or more target sequences of the probe set.

Primers based on the nucleotide sequences of target sequences can be designed for use in amplification of the target sequences. For use in amplification reactions such as PCR, a pair of primers can be used. The exact composition of the primer sequences is not necessarily critical, but for most applications the primers may hybridize to specific sequences of the probe set under stringent conditions, particularly under conditions of high stringency, as known in the art. The pairs of primers are usually chosen so as to generate an amplification product of at least about 50 nucleotides, more usually at least about 100 nucleotides. Algorithms for the selection of primer sequences are generally known, and are available in commercial software packages. These primers may be used in standard quantitative or qualitative PCR-based assays to assess transcript expression levels of RNAs defined by the probe set. In some instances, these primers may be used in combination with probes, such as molecular beacons in amplifications using real-time PCR.

In one embodiment, the primers or primer pairs, when used in an amplification reaction, specifically amplify at least a portion of a nucleic acid sequence of a target selected from Table 2 and/or Table 5 (or subgroups thereof as set forth herein), an RNA form thereof, or a complement to either thereof.

A label can optionally be attached to or incorporated into a probe or primer polynucleotide to allow detection and/or quantitation of a target polynucleotide representing the target sequence of interest. The target polynucleotide may be the expressed target sequence RNA itself, a cDNA copy thereof, or an amplification product derived therefrom, and may be the positive or negative strand, so long as it can be specifically detected in the assay being used. Similarly, an antibody may be labeled.

In certain multiplex formats, labels used for detecting different targets may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g. biotin-avidin or streptavidin). Many labels are commercially available in activated forms which can readily be used for such conjugation (for example through amine acylation), or labels may be attached through known or determinable conjugation schemes, many of which are known in the art.

Labels useful in the methods, compositions, systems and kits described herein include any substance which can be detected when bound to or incorporated into the biomolecule of interest. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof. Quenching schemes may be used, wherein a quencher and a fluorophore as members of a quenching pair may be used on a probe, such that a change in optical parameters occurs upon binding to the target introduce or quench the signal from the fluorophore. One example of such a system is a molecular beacon. Suitable quencher/fluorophore systems are known in the art. The label may be bound through a variety of intermediate linkages. For example, a polynucleotide may comprise a biotin-binding species, and an optically detectable label may be conjugated to biotin and then bound to the labeled polynucleotide. Similarly, a polynucleotide sensor may comprise an immunological species such as an antibody or fragment, and a secondary antibody containing an optically detectable label may be added.

Chromophores useful in the methods described herein include any substance which can absorb energy and emit light. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes and green fluorescent protein.

In some embodiments, polynucleotides of the compositions, methods, systems and kits described herein comprise at least 20 consecutive bases of the nucleic acid sequence of a target selected from Table 2 and/or Table 5 or a complement thereto. The polynucleotides may comprise at least 21, 22, 23, 24, 25, 27, 30, 32, 35, 40, 45, 50, or more consecutive bases of the nucleic acids sequence of a target selected from Table 2 and/or Table 5, as applicable.

The polynucleotides may be provided in a variety of formats, including as solids, in solution, or in an array. The polynucleotides may optionally comprise one or more labels, which may be chemically and/or enzymatically incorporated into the polynucleotide.

In some embodiments, one or more polynucleotides provided herein can be provided on a substrate. The substrate can comprise a wide range of material, either biological, non-biological, organic, inorganic, or a combination of any of these. For example, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonates, or combinations thereof. Conducting polymers and photoconductive materials can be used.

The substrate can take the form of an array, a photodiode, an optoelectronic sensor such as an optoelectronic semiconductor chip or optoelectronic thin-film semiconductor, or a biochip. The location(s) of probe(s) on the substrate can be addressable; this can be done in highly dense formats, and the location(s) can be microaddressable or nanoaddressable.

### Diagnostic Samples

A biological sample is collected from a subject in need of treatment for cancer to evaluate whether a patient will benefit from neoadjuvant chemotherapy. Diagnostic samples for use with the systems and in the methods of the present disclosure comprise nucleic acids suitable for providing RNAs expression information. In principle, the biological sample from which the expressed RNA is obtained and analyzed for target sequence expression can be any material suspected of comprising cancerous bladder tissue or cells. The diagnostic sample can be a biological sample used directly in a method of the disclosure. In some instances, the diagnostic sample can be a sample prepared from a biological sample.

In one embodiment, the sample or portion of the sample comprising or suspected of comprising cancerous tissue or cells can be any source of biological material, including cells, tissue or fluid, including bodily fluids. Non-limiting examples of the source of the sample include an aspirate, a needle biopsy, a cytology pellet, a bulk tissue preparation or a section thereof obtained for example by surgery or autopsy, lymph fluid, blood, plasma, serum, tumors, and organs. In some embodiments, the sample is from urine comprising cancerous cells. In some instances, the sample is from a bladder tumor biopsy.

The samples may be archival samples, having a known and documented medical outcome, or may be samples from current patients whose ultimate medical outcome is not yet known.

In some embodiments, the sample may be dissected prior to molecular analysis. The sample may be prepared via macrodissection of a bulk tumor specimen or portion thereof, or may be treated via microdissection, for example via Laser Capture Microdissection (LCM).

The sample may initially be provided in a variety of states, as fresh tissue, fresh frozen tissue, fine needle aspirates, and may be fixed or unfixed. Frequently, medical laboratories routinely prepare medical samples in a fixed state, which facilitates tissue storage. A variety of fixatives can be used to fix tissue to stabilize the morphology of cells, and may be used alone or in combination with other agents. Exemplary fixatives include crosslinking agents, alcohols, acetone, Bouin's solution, Zenker solution, Helv solution, osmic acid solution and Carnoy solution.

Crosslinking fixatives can comprise any agent suitable for forming two or more covalent bonds, for example an aldehyde. Sources of aldehydes typically used for fixation include formaldehyde, paraformaldehyde, glutaraldehyde or formalin. Preferably, the crosslinking agent comprises formaldehyde, which may be included in its native form or in the form of paraformaldehyde or formalin. One of skill in the art would appreciate that for samples in which crosslinking fixatives have been used special preparatory steps may be necessary including for example heating steps and proteinase-k digestion; see methods.

One or more alcohols may be used to fix tissue, alone or in combination with other fixatives. Exemplary alcohols used for fixation include methanol, ethanol and isopropanol.

Formalin fixation is frequently used in medical laboratories. Formalin comprises both an alcohol, typically methanol, and formaldehyde, both of which can act to fix a biological sample.

Whether fixed or unfixed, the biological sample may optionally be embedded in an embedding medium. Exemplary embedding media used in histology including paraffin, Tissue-Tek® V.I.P.™, Paramat, Paramat Extra, Paraplast, Paraplast X-tra, Paraplast Plus, Peel Away Paraffin Embedding Wax, Polyester Wax, Carbowax Polyethylene Glycol, Polyfin™, Tissue Freezing Medium TFMFM, Cryo-Gef™, and OCT Compound (Electron Microscopy Sciences. Hatfield, Pa.). Prior to molecular analysis, the embedding material may be removed via any suitable techniques, as known in the art. For example, where the sample is embedded in wax, the embedding material may be removed by extraction with organic solvent(s), for example xylenes. Kits are commercially available for removing embedding media from tissues. Samples or sections thereof may be subjected to further processing steps as needed, for example serial hydration or dehydration steps.

In some embodiments, the sample is a fixed, wax-embedded biological sample. Frequently, samples from medical laboratories are provided as fixed, wax-embedded samples, most commonly as formalin-fixed, paraffin embedded (FFPE) tissues.

Whatever the source of the biological sample, the target polynucleotide that is ultimately assayed can be prepared synthetically (in the case of control sequences), but typically is purified from the biological source and subjected to one or more preparative steps. The RNA may be purified to remove or diminish one or more undesired components from the biological sample or to concentrate it. Conversely, where the RNA is too concentrated for the particular assay, it may be diluted.

### RNA Extraction

RNA can be extracted and purified from biological samples using any suitable technique. A number of techniques are known in the art, and several are commercially available (e.g., FormaPure nucleic acid extraction kit, Agencourt Biosciences, Beverly Mass., High Pure FFPE RNA Micro Kit, Roche Applied Science, Indianapolis, Ind.). RNA can be extracted from frozen tissue sections using TRIzol (Invitrogen, Carlsbad, Calif.) and purified using RNeasy Protect kit (Qiagen, Valencia, Calif.). RNA can be further purified using DNAse I treatment (Ambion, Austin, Tex.) to eliminate any contaminating DNA. RNA concentrations can be made using a Nanodrop ND-1000 spectrophotometer (Nanodrop Technologies, Rockland, Del.). RNA can be further purified to eliminate contaminants that interfere with cDNA synthesis by cold sodium acetate precipitation. RNA integrity can be evaluated by running electropherograms, and RNA integrity number (RIN, a correlative measure that indicates intactness of mRNA) can be determined using the RNA 6000 PicoAssay for the Bioanalyzer 2100 (Agilent Technologies, Santa Clara, Calif.).

### Kits

Kits for performing the desired method(s) are also provided, and comprise a container or housing for holding the components of the kit, one or more vessels containing one or more nucleic acid(s), and optionally one or more vessels containing one or more reagents. The reagents include those described in the sections above, and those reagents useful for performing the methods described, including amplification reagents, and may include one or more probes, primers or primer pairs, enzymes (including polymerases and ligases), intercalating dyes, labeled probes, and labels that can be incorporated into amplification products.

In some embodiments, the kit comprises primers or primer pairs specific for those subsets and combinations of target sequences described herein. The primers or pairs of primers are suitable for selectively amplifying the target sequences. The kit may comprise at least two, three, four or five primers or pairs of primers suitable for selectively amplifying one or more targets. The kit may comprise at least 5, 10, 15, 20, 30, 40, 50, 60, 70, 80, 90, 100, 110, or more primers or pairs of primers suitable for selectively amplifying one or more targets.

In some embodiments, the primers or primer pairs of the kit, when used in an amplification reaction, specifically amplify a non-coding target, coding target, exonic, or non-exonic target described herein, a nucleic acid sequence corresponding to a target selected from Table 2 and/or Table 5, an RNA form thereof, or a complement to either thereof. The kit may include a plurality of such primers or primer pairs which can specifically amplify a corresponding plurality of different amplify a non-coding target, coding target, exonic, or non-exonic transcript described herein, a nucleic acid sequence corresponding to a target selected from Table 2 and/or Table 5, RNA forms thereof, or complements thereto. At least two, three, four or five primers or pairs of primers suitable for selectively amplifying the one or more targets can be provided in kit form. In some embodiments, the kit comprises from five to fifty primers or pairs of primers suitable for amplifying the one or more targets.

The reagents may independently be in liquid or solid form. The reagents may be provided in mixtures. Control samples and/or nucleic acids may optionally be provided in the kit. Control samples may include tissue and/or nucleic acids obtained from or representative of tumor samples from patients showing no evidence of disease, as well as tissue and/or nucleic acids obtained from or representative of tumor samples from patients that develop systemic cancer.

The nucleic acids may be provided in an array format, and thus an array or microarray may be included in the kit. The kit optionally may be certified by a government agency for use in prognosing the disease outcome of cancer patients and/or for designating a treatment modality.

Instructions for using the kit to perform one or more methods of the disclosure can be provided with the container, and can be provided in any fixed medium. The instructions may be located inside or outside the container or housing, and/or may be printed on the interior or exterior of any surface thereof. A kit may be in multiplex form for concurrently detecting and/or quantitating one or more different target polynucleotides representing the expressed target sequences.

### Amplification and Hybridization

Following sample collection and nucleic acid extraction, the nucleic acid portion of the sample comprising RNA that is or can be used to prepare the target polynucleotide(s) of interest can be subjected to one or more preparative reactions. These preparative reactions can include in vitro transcription (IVT), labeling, fragmentation, amplification and other reactions. The mRNA can first be treated with reverse transcriptase and a primer to create cDNA prior to detection, quantitation and/or amplification; this can be done in vitro with purified mRNA or in situ, e.g., in cells or tissues affixed to a slide.

By “amplification” is meant any process of producing at least one copy of a nucleic acid, in this case an expressed RNA, and in many cases produces multiple copies. An amplification product can be RNA or DNA, and may include a complementary strand to the expressed target sequence. DNA amplification products can be produced initially through reverse translation and then optionally from further amplification reactions. The amplification product may include all or a portion of a target sequence, and may optionally be labeled. A variety of amplification methods are suitable for use, including polymerase-based methods and ligation-based methods. Exemplary amplification techniques include the polymerase chain reaction method (PCR), the lipase chain reaction (LCR), ribozyme-based methods, self-sustained sequence replication (3SR), nucleic acid sequence-based amplification (NASBA), and the use of Q Beta replicase, reverse transcription, nick translation, and the like.

Asymmetric amplification reactions may be used to preferentially amplify one strand representing the target sequence that is used for detection as the target polynucleotide. In some cases, the presence and/or amount of the amplification product itself may be used to determine the expression level of a given target sequence. In some instances, the amplification product may be used to hybridize to an array or other substrate comprising sensor polynucleotides which are used to detect and/or quantitate target sequence expression.

The first cycle of amplification in polymerase-based methods typically forms a primer extension product complementary to the template strand. If the template is single-stranded RNA, a polymerase with reverse transcriptase activity is used in the first amplification to reverse transcribe the RNA to DNA, and additional amplification cycles can be performed to copy the primer extension products. The primers for a PCR must, of course, be designed to hybridize to regions in their corresponding template that can produce an amplifiable segment; thus, each primer must hybridize so that its 3′ nucleotide is paired to a nucleotide in its complementary template strand that is located 3′ from the 3′ nucleotide of the primer used to replicate that complementary template strand in the PCR

The target polynucleotide can be amplified by contacting one or more strands of the target polynucleotide with a primer and a polymerase having suitable activity to extend the primer and copy the target polynucleotide to produce a full-length complementary polynucleotide or a smaller portion thereof. Any enzyme having a polymerase activity that can copy the target polynucleotide can be used, including DNA polymerases, RNA polymerases, reverse transcriptases, and enzymes having more than one type of polymerase or enzyme activity. The enzyme can be thermolabile or thermostable. Mixtures of enzymes can also be used. Exemplary enzymes include: DNA polymerases such as DNA Polymerase I (“Pol I”), the Klenow fragment of Pol I, T4, T7, Sequenase® T7, Sequenase® Version 2.0 T7, Tub, Taq, Tth, Pfic, Pfu, Tsp, Tfl, Tli and Pyrococcus sp GB-D DNA polymerases; RNA polymerases such as F. coil, SP6, T3 and T7 RNA polymerases; and reverse transcriptases such as AMV, M-MuLV, MMLV, RNAse H MMLV (SuperScript®), SuperScript® II, ThermoScript®, HIV-1, and RAV2 reverse transcriptases. All of these enzymes are commercially available. Exemplary polymerases with multiple specificities include RAV2 and Tli (exo-) polymerases. Exemplary thermostable polymerases include Tub, Taq, Tth, Pfic, Pfi, Tsp, Tfl, Tli and Pyrococcus sp. GB-D DNA polymerases.

Suitable reaction conditions are chosen to permit amplification of the target polynucleotide, including pH, buffer, ionic strength, presence and concentration of one or more salts, presence and concentration of reactants and cofactors such as nucleotides and magnesium and/or other metal ions (e.g., manganese), optional cosolvents, temperature, thermal cycling profile for amplification schemes comprising a polymerase chain reaction, and may depend in part on the polymerase being used as well as the nature of the sample. Cosolvents include formamide (typically at from about 2 to about 10%), glycerol (typically at from about 5 to about 10%), and DMSO (typically at from about 0.9 to about 10%). Techniques may be used in the amplification scheme in order to minimize the production of false positives or artifacts produced during amplification. These include “touchdown” PCR, hot-start techniques, use of nested primers, or designing PCR primers so that they form stem-loop structures in the event of primer-dimer formation and thus are not amplified. Techniques to accelerate PCR can be used, for example centrifugal PCR, which allows for greater convection within the sample, and comprising infrared heating steps for rapid heating and cooling of the sample. One or more cycles of amplification can be performed. An excess of one primer can be used to produce an excess of one primer extension product during PCR; preferably, the primer extension product produced in excess is the amplification product to be detected. A plurality of different primers may be used to amplify different target polynucleotides or different regions of a particular target polynucleotide within the sample.

An amplification reaction can be performed under conditions which allow an optionally labeled sensor polynucleotide to hybridize to the amplification product during at least part of an amplification cycle. When the assay is performed in this manner, real-time detection of this hybridization event can take place by monitoring for light emission or fluorescence during amplification, as known in the art.

Where the amplification product is to be used for hybridization to an array or microarray, a number of suitable commercially available amplification products are available. These include amplification kits available from NuGEN, Inc. (San Carlos, Calif.), including the WT-Ovation™ System, WT-Ovation™ System v2, WT-Ovation™ Pico System, WT-Ovation™ FFPE Exon Module, WT-Ovation™ FFPE Exon Module RiboAmp and RiboAmp ^{Plus} RNA Amplification Kits (MDS Analytical Technologies (formerly Arcturus) (Mountain View, Calif.), Genisphere, Inc. (Hatfield, Pa.), including the RampUp Plus™ and SenseAmp™ RNA Amplification kits, alone or in combination. Amplified nucleic acids may be subjected to one or more purification reactions after amplification and labeling, for example using magnetic beads (e.g., RNACIean magnetic beads, Agencourt Biosciences).

Multiple RNA biomarkers can be analyzed using real-time quantitative multiplex RT-PCR platforms and other multiplexing technologies such as GenomeLab GeXP Genetic Analysis System (Beckman Coulter, Foster City, Calif.), SmartCycler® 9600 or GeneXpert® Systems (Cepheid, Sunnyvale, Calif.), ABI 7900 HT Fast Real Time PCR system (Applied Biosystems, Foster City, Calif.), LightCycler® 480 System (Roche Molecular Systems, Pleasanton, Calif.), xMAP 100 System (Luminex, Austin, Tex.) Solexa Genome Analysis System (Illumina, Hayward, Calif.), OpenArray Real Time qPCR (BioTrove, Woburn, Mass.) and BeadXpress System (Illumina, Hayward, Calif.).

Detection and/or Quantification of Target Sequences

Any method of detecting and/or quantitating the expression of the encoded target sequences can in principle be used in the methods, compositions, systems and kits described herein. The expressed target sequences can be directly detected and/or quantitated, or may be copied and/or amplified to allow detection of amplified copies of the expressed target sequences or its complement.

Methods for detecting and/or quantifying a target can include Northern blotting, sequencing, array or microarray hybridization, serial analysis of gene expression (SAGE), by enzymatic cleavage of specific structures (e.g., an Invader® assay, Third Wave Technologies, e.g. as described in U.S. Pat. Nos. 5,846,717, 6,090,543; 6,001,567; 5,985,557; and 5,994,069) and amplification methods, e.g. RT-PCR, including in a TaqMan® assay (PE Biosystems, Foster City, Calif., e.g. as described in U.S. Pat. Nos. 5,962,233 and 5,538,848), and may be quantitative or semi-quantitative, and may vary depending on the origin, amount and condition of the available biological sample. Combinations of these methods may also be used. For example, nucleic acids may be amplified, labeled and subjected to microarray analysis.

In some instances, target sequences may be detected by sequencing. Sequencing methods may comprise whole genome sequencing or exome sequencing. Sequencing methods such as Maxim-Gilbert, chain-termination, or high-throughput systems may also be used. Additional, suitable sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, and SOLiD sequencing.

Additional methods for detecting and/or quantifying a target include single-molecule sequencing (e.g., Helicos, PacBio), sequencing by synthesis (e.g., Illumina, Ion Torrent), sequencing by ligation (e.g., ABI SOLID), sequencing by hybridization (e.g., Complete Genomics), in situ hybridization, bead-array technologies (e.g., Luminex xMAP, Illumina BeadChips), branched DNA technology (e.g., Panomics, Genisphere). Sequencing methods may use fluorescent (e.g., Illumina) or electronic (e.g., Ion Torrent, Oxford Nanopore) methods of detecting nucleotides.

### Reverse Transcription for ORT-PCR Analysis

Reverse transcription can be performed by any method known in the art. For example, reverse transcription may be performed using the Omniscript kit (Qiagen, Valencia, Calif.), Superscript III kit (Invitrogen, Carlsbad, Calif.), for RT-PCR. Target-specific priming can be performed in order to increase the sensitivity of detection of target sequences and generate target-specific cDNA.

### TaqMan® Gene Expression Analysis

TaqMan® RT-PCR can be performed using Applied Biosystems Prism (ABI) 7900 HT instruments in a 5 1.11 volume with target sequence-specific cDNA equivalent to 1 ng total RNA.

Primers and probes concentrations for TaqMan analysis are added to amplify fluorescent amplicons using PCR cycling conditions such as 95° C. for 10 minutes for one cycle, 95° C. for 20 seconds, and 60° C. for 45 seconds for 40 cycles. A reference sample can be assayed to ensure reagent and process stability. Negative controls (e.g., no template) should be assayed to monitor any exogenous nucleic acid contamination.

### Classification Arrays

The present disclosure contemplates that a probe set or probes derived therefrom may be provided in an array format. In the context of the present disclosure, an “array” is a spatially or logically organized collection of polynucleotide probes. An array comprising probes specific for a coding target, non-coding target, or a combination thereof may be used. In some instances, an array comprising probes specific for two or more of transcripts of a target selected from Table 2 and/or Table 5, or a product derived thereof, can be used. Desirably, an array may be specific for 5, 10, 15, 20, 25, 30 or more of transcripts of a target selected from Table 2 and/or Table 5. Expression of these sequences may be detected alone or in combination with other transcripts. In some embodiments, an array is used which comprises a wide range of sensor probes for bladder-specific expression products, along with appropriate control sequences. In some instances, the array may comprise the Human Exon 1.0 ST Array (HuEx 1.0 ST, Affymetrix, Inc., Santa Clara, Calif.).

Typically the polynucleotide probes are attached to a solid substrate and are ordered so that the location (on the substrate) and the identity of each are known. The polynucleotide probes can be attached to one of a variety of solid substrates capable of withstanding the reagents and conditions necessary for use of the array. Examples include, but are not limited to, polymers, such as (poly)tetrafluoroethylene, (poly)vinylidenedifluoride, polystyrene, polycarbonate, polypropylene and polystyrene; ceramic; silicon; silicon dioxide; modified silicon; (fused) silica, quartz or glass; functionalized glass; paper, such as filter paper; diazotized cellulose; nitrocellulose filter; nylon membrane; and polyacrylamide gel pad. Substrates that are transparent to light are useful for arrays that may be used in an assay that involves optical detection.

Examples of array formats include membrane or filter arrays (for example, nitrocellulose, nylon arrays), plate arrays (for example, multiwell, such as a 24-, 96-, 256-, 384-, 864- or 1536-well, microtitre plate arrays), pin arrays, and bead arrays (for example, in a liquid “slurry”). Arrays on substrates such as glass or ceramic slides are often referred to as chip arrays or “chips.” Such arrays are well known in the art. In one embodiment of the present disclosure, the Cancer Prognosticarray is a chip.

### Data Analysis

In some embodiments, one or more pattern recognition methods can be used in analyzing the expression level of target sequences. The pattern recognition method can comprise a linear combination of expression levels, or a nonlinear combination of expression levels. In some embodiments, expression measurements for RNA transcripts or combinations of RNA transcript levels are formulated into linear or non-linear models or algorithms (e.g., an ‘expression signature’) and converted into a likelihood score. This likelihood score may indicate the probability that a biological sample is from a patient who will benefit from neoadjuvant chemotherapy. Additionally, a likelihood score may indicate the probability that a biological sample is from a patient who may exhibit no evidence of disease, who may exhibit systemic cancer, or who may exhibit biochemical recurrence. The likelihood score can be used to distinguish these disease states. The models and/or algorithms can be provided in machine readable format, and may be used to correlate expression levels or an expression profile with a disease state, and/or to designate a treatment modality for a patient or class of patients.

Assaying the expression level for a plurality of targets may comprise the use of an algorithm or classifier. Array data can be managed, classified, and analyzed using techniques known in the art. Assaying the expression level for a plurality of targets may comprise probe set modeling and data pre-processing. Probe set modeling and data pre-processing can be derived using the Robust Multi-Array (RMA) algorithm or variants GC-RMA,JRMA, Probe Logarithmic Intensity Error (PLIER) algorithm, or variant iterPLIER, or Single-Channel Array Normalization (SCAN) algorithm. Variance or intensity filters can be applied to pre-process data using the RMA algorithm, for example by removing target sequences with a standard deviation of <10 or a mean intensity of <100 intensity units of a normalized data range, respectively.

In some instances, assaying the expression level for a plurality of targets may comprise the use of a machine learning algorithm. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). In some instances, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. In some instances, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. In some instances, the machine learning algorithm may comprise Data Pre-processing.

Preferably, the machine learning algorithms may include, but are not limited to, Average One-Dependence Estimators (AODE), Fisher's linear discriminant, Logistic regression, Perceptron, Multilayer Perceptron, Artificial Neural Networks, Support vector machines, Quadratic classifiers, Boosting, Decision trees, C4.5, Bayesian networks, Hidden Markov models, High-Dimensional Discriminant Analysis, and Gaussian Mixture Models. The machine learning algorithm may comprise support vector machines, Naïve Bayes classifier, k-nearest neighbor, high-dimensional discriminant analysis, or Gaussian mixture models. In some instances, the machine learning algorithm comprises Random Forests.

### Subtyping

Molecular subtyping is a method of classifying bladder cancer into one of multiple genetically-distinct categories, or subtypes. Each subtype responds differently to different kinds of treatments, and the presence of a particular subtype is predictive of, for example, chemoresistance, higher risk of recurrence, or good or poor prognosis for an individual. The inventors of the present disclosure discovered that classification of bladder cancer into five subtypes, including, basal/squamous, luminal, luminal-infiltrated, luminal-papillary, and neuronal subtypes is clinically useful for predicting patient outcome and response to anti-cancer therapy (see Examples). As described herein, each subtype has a unique molecular and clinical fingerprint. Differential expression analysis of one or more of the gene targets listed in Table 2 and/or Table 5 allows for the identification of the molecular subtype of a bladder cancer. In some instances, the molecular subtyping methods of the present disclosure are used in combination with other biomarkers, like tumor grade and hormone levels, for analyzing the bladder cancer.

### Prognosis and Prediction of Treatment Response to Anti-Cancer Therapy

Bladder cancer subtyping can be utilized to predict outcome and whether or not a cancer patient will benefit from certain anti-cancer therapy (e.g., neoadjuvant chemotherapy). For example, patients with luminal-papillary bladder cancer have the best prognosis. FGFR3+ tumors have better survival. Patients with basal/squamous and luminal tumors have a worse prognosis than those with luminal-papillary tumors. Patients with luminal-infiltrated and neuronal tumors tumors had the worst prognosis. Patients with FGFR3+ luminal tumors have better survival than other patients. Such patients could be offered less invasive treatment and may be candidates for treatment with FGFR3-inhibitors instead of neoadjuvant chemotherapy.

### Therapeutic Regimens

Diagnosing, predicting, or monitoring a status or outcome of bladder cancer may comprise treating the bladder cancer or preventing cancer progression. In addition, diagnosing, predicting, or monitoring a status or outcome of bladder cancer may comprise identifying or predicting which patients will be responders or non-responders to an anti-cancer therapy (e.g., neoadjuvant chemotherapy). In some instances, diagnosing, predicting, or monitoring may comprise determining a therapeutic regimen. Determining a therapeutic regimen may comprise administering an anti-cancer therapy. In some instances, determining a therapeutic regimen may comprise modifying, recommending, continuing or discontinuing an anti-cancer regimen. In some instances, if the sample expression patterns are consistent with the expression pattern for a known disease or disease outcome, the expression patterns can be used to designate one or more treatment modalities (e.g., therapeutic regimens, such as neoadjuvant chemotherapy or other anti-cancer regimen). An anti-cancer regimen may comprise one or more anti-cancer therapies. Examples of anti-cancer therapies include surgery, chemotherapy, radiation therapy, immunotherapy/biological therapy, and photodynamic therapy.

For example, a patient is selected for treatment with neoadjuvant chemotherapy if the patient is identified as being likely to be responsive to neoadjuvant chemotherapy based on subtyping of the bladder cancer, as described herein. Neoadjuvant chemotherapy may be performed prior to other anti-cancer treatments such as, but not limited to, surgery (e.g., transurethral resection or cystectomy), radiation therapy, immunotherapy (e.g., Bacillus Calmette-Guerin (BCG) or anti-PDL 1 immunotherapy), hormonal therapy, biologic therapy, or any combination thereof. Patients, especially those not identified as likely to benefit from neoadjuvant chemotherapy, may omit neoadjuvant chemotherapy and instead be administered other cancer treatments directly.

Examples of chemotherapeutic agents that may be used in treating bladder cancer include alkylating agents, anti-metabolites, plant alkaloids and terpenoids, vinca alkaloids, podophyllotoxin, taxanes, topoisomerase inhibitors, and cytotoxic antibiotics. Cisplatin, carboplatin, and oxaliplatin are examples of alkylating agents. Other alkylating agents include mechlorethamine, cyclophosphamide, chlorambucil, ifosfamide. Alkylating agents may impair cell function by forming covalent bonds with the amino, carboxyl, sulfhydryl, and phosphate groups in biologically important molecules. In some instances, alkylating agents may chemically modify a cell's DNA.

In some embodiments, the subject has a bladder cancer that is determined to be FGFR3 positive. Such subjects could be offered less invasive treatment such as, for example, treatment with Erdafitinib, an FGFR-inhibitor. Consequently, FGFR3+ cancer subjects are candidates for treatment with FGFR3-inhibitors instead of neoadjuvant chemotherapy (NAC), as patients with luminal tumors may benefit less from NAC while still being exposed to chemotherapy-related toxicity.

Anti-metabolites are another example of chemotherapeutic agents. Anti-metabolites may masquerade as purines or pyrimidines and may prevent purines and pyrimidines from becoming incorporated in to DNA during the “S” phase (of the cell cycle), thereby stopping normal development and division. Antimetabolites may also affect RNA synthesis. Examples of metabolites include azathioprine and mercaptopurine.

Alkaloids may be derived from plants and block cell division may also be used for the treatment of cancer. Alkyloids may prevent microtubule function. Examples of alkaloids are vinca alkaloids and taxanes. Vinca alkaloids may bind to specific sites on tubulin and inhibit the assembly of tubulin into microtubules (M phase of the cell cycle). The vinca alkaloids may be derived from the Madagascar periwinkle, Catharanthus roseus (formerly known as Vinca rosea). Examples of vinca alkaloids include, but are not limited to, vincristine, vinblastine, vinorelbine, or vindesine. Taxanes are diterpenes produced by the plants of the genus Taxus (yews). Taxanes may be derived from natural sources or synthesized artificially. Taxanes include paclitaxel (Taxol) and docetaxel (Taxotere). Taxanes may disrupt microtubule function. Microtubules are essential to cell division, and taxanes may stabilize GDP-bound tubulin in the microtubule, thereby inhibiting the process of cell division. Thus, in essence, taxanes may be mitotic inhibitors. Taxanes may also be radiosensitizing and often contain numerous chiral centers.

Alternative chemotherapeutic agents include podophyllotoxin. Podophyllotoxin is a plant-derived compound that may help with digestion and may be used to produce cytostatic drugs such as etoposide and teniposide. They may prevent the cell from entering the G1 phase (the start of DNA replication) and the replication of DNA (the S phase).

Topoisomerases are essential enzymes that maintain the topology of DNA. Inhibition of type I or type 11 topoisomerases may interfere with both transcription and replication of DNA by upsetting proper DNA supercoiling. Some chemotherapeutic agents may inhibit topoisomerases.

For example, some type I topoisomerase inhibitors include camptothecins: irinotecan and topotecan. Examples of type 11 inhibitors include amsacrine, etoposide, etoposide phosphate, and teniposide.

Another example of chemotherapeutic agents is cytotoxic antibiotics. Cytotoxic antibiotics are a group of antibiotics that are used for the treatment of cancer because they may interfere with DNA replication and/or protein synthesis. Cytotoxic antibiotics include, but are not limited to, actinomycin, anthracyclines, doxorubicin, daunorubicin, valrubicin, idarubicin, epirubicin, bleomycin, plicamycin, and mitomycin.

Surgical oncology uses surgical methods to diagnose, stage, and treat cancer, and to relieve certain cancer-related symptoms. Surgery may be used to remove the tumor (e.g., excisions, resections, debulking surgery), reconstruct a part of the body (e.g., restorative surgery), and/or to relieve symptoms such as pain (e.g., palliative surgery). Surgery may also include cryosurgery. Cryosurgery (also called cryotherapy) may use extreme cold produced by liquid nitrogen (or argon gas) to destroy abnormal tissue. Cryosurgery can be used to treat external tumors, such as those on the skin. For external tumors, liquid nitrogen can be applied directly to the cancer cells with a cotton swab or spraying device. Cryosurgery may also be used to treat tumors inside the body (internal tumors and tumors in the bone). For internal tumors, liquid nitrogen or argon gas may be circulated through a hollow instrument called a cryoprobe, which is placed in contact with the tumor. An ultrasound or MRI may be used to guide the cryoprobe and monitor the freezing of the cells, thus limiting damage to nearby healthy tissue. A ball of ice crystals may form around the probe, freezing nearby cells. Sometimes more than one probe is used to deliver the liquid nitrogen to various parts of the tumor. The probes may be put into the tumor during surgery or through the skin (percutaneously). After cryosurgery, the frozen tissue thaws and may be naturally absorbed by the body (for internal tumors), or may dissolve and form a scab (for external tumors).

In some instances, the anti-cancer treatment may comprise radiation therapy. Radiation can come from a machine outside the body (external-beam radiation therapy) or from radioactive material placed in the body near cancer cells (internal radiation therapy, more commonly called brachytherapy). In some embodiments, the anti-cancer treatment may comprise an FGFR3-inhibitor. Systemic radiation therapy uses a radioactive substance, given by mouth or into a vein that travels in the blood to tissues throughout the body.

External-beam radiation therapy may be delivered in the form of photon beams (either x-rays or gamma rays). A photon is the basic unit of light and other forms of electromagnetic radiation. An example of external-beam radiation therapy is called 3-dimensional conformal radiation therapy (3D-CRT). 3D-CRT may use computer software and advanced treatment machines to deliver radiation to very precisely shaped target areas. Many other methods of external-beam radiation therapy are currently being tested and used in cancer treatment. These methods include, but are not limited to, intensity-modulated radiation therapy (IMRT), image-guided radiation therapy (IGRT), Stereotactic radiosurgery (SRS), Stereotactic body radiation therapy (SBRT), and proton therapy.

Intensity-modulated radiation therapy (IMRT) is an example of external-beam radiation and may use hundreds of tiny radiation beam-shaping devices, called collimators, to deliver a single dose of radiation. The collimators can be stationary or can move during treatment, allowing the intensity of the radiation beams to change during treatment sessions. This kind of dose modulation allows different areas of a tumor or nearby tissues to receive different doses of radiation. IMRT is planned in reverse (called inverse treatment planning). In inverse treatment planning, the radiation doses to different areas of the tumor and surrounding tissue are planned in advance, and then a high-powered computer program calculates the required number of beams and angles of the radiation treatment. In contrast, during traditional (forward) treatment planning, the number and angles of the radiation beams are chosen in advance and computers calculate how much dose may be delivered from each of the planned beams. The goal of IMRT is to increase the radiation dose to the areas that need it and reduce radiation exposure to specific sensitive areas of surrounding normal tissue.

Another example of external-beam radiation is image-guided radiation therapy (IGRT). In IGRT, repeated imaging scans (CT, MRJ, or PET) may be performed during treatment. These imaging scans may be processed by computers to identify changes in a tumor's size and location due to treatment and to allow the position of the patient or the planned radiation dose to be adjusted during treatment as needed. Repeated imaging can increase the accuracy of radiation treatment and may allow reductions in the planned volume of tissue to be treated, thereby decreasing the total radiation dose to normal tissue.

Tomotherapy is a type of image-guided IMRT. A tomotherapy machine is a hybrid between a CT imaging scanner and an external-beam radiation therapy machine. The part of the tomotherapy machine that delivers radiation for both imaging and treatment can rotate completely around the patient in the same manner as a normal CT scanner. Tomotherapy machines can capture CT images of the patient's tumor immediately before treatment sessions, to allow for very precise tumor targeting and sparing of normal tissue.

Stereotactic radiosurgery (SRS) can deliver one or more high doses of radiation to a small tumor. SRS uses extremely accurate image-guided tumor targeting and patient positioning. Therefore, a high dose of radiation can be given without excess damage to normal tissue. SRS can be used to treat small tumors with well-defined edges. It is most commonly used in the treatment of brain or spinal tumors and brain metastases from other cancer types. For the treatment of some brain metastases, patients may receive radiation therapy to the entire brain (called whole-brain radiation therapy) in addition to SRS. SRS requires the use of a head frame or other device to immobilize the patient during treatment to ensure that the high dose of radiation is delivered accurately.

Stereotactic body radiation therapy (SBRT) delivers radiation therapy in fewer sessions, using smaller radiation fields and higher doses than 3D-CRT in most cases. SBRT may treat tumors that lie outside the brain and spinal cord. Because these tumors are more likely to move with the normal motion of the body, and therefore cannot be targeted as accurately as tumors within the brain or spine, SBRT is usually given in more than one dose. SBRT can be used to treat small, isolated tumors, including cancers in the lung and liver. SBRT systems may be known by their brand names, such as the CyberKnife®.

In proton therapy, external-beam radiation therapy may be delivered by proton. Protons are a type of charged particle. Proton beams differ from photon beams mainly in the way they deposit energy in living tissue. Whereas photons deposit energy in small packets all along their path through tissue, protons deposit much of their energy at the end of their path (called the Bragg peak) and deposit less energy along the way. Use of protons may reduce the exposure of normal tissue to radiation, possibly allowing the delivery of higher doses of radiation to a tumor.

Other charged particle beams such as electron beams may be used to irradiate superficial tumors, such as skin cancer or tumors near the surface of the body, but they cannot travel very far through tissue.

Internal radiation therapy (brachytherapy) is radiation delivered from radiation sources (radioactive materials) placed inside or on the body. Several brachytherapy techniques are used in cancer treatment. Interstitial brachytherapy may use a radiation source placed within tumor tissue, such as within a bladder tumor. Intracavitary brachytherapy may use a source placed within a surgical cavity or a body cavity, such as the chest cavity, near a tumor. Episcleral brachytherapy, which may be used to treat melanoma inside the eye, may use a source that is attached to the eye. In brachytherapy, radioactive isotopes can be sealed in tiny pellets or “seeds.” These seeds may be placed in patients using delivery devices, such as needles, catheters, or some other type of carrier. As the isotopes decay naturally, they give off radiation that may damage nearby cancer cells. Brachytherapy may be able to deliver higher doses of radiation to some cancers than external-beam radiation therapy while causing less damage to normal tissue.

Brachytherapy can be given as a low-dose-rate or a high-dose-rate treatment. In low-dose-rate treatment, cancer cells receive continuous low-dose radiation from the source over a period of several days. In high-dose-rate treatment, a robotic machine attached to delivery tubes placed inside the body may guide one or more radioactive sources into or near a tumor, and then removes the sources at the end of each treatment session. High-dose-rate treatment can be given in one or more treatment sessions. An example of a high-dose-rate treatment is the MammoSite® system.

The placement of brachytherapy sources can be temporary or permanent. For permanent brachytherapy, the sources may be surgically sealed within the body and left there, even after all of the radiation has been given off. In some instances, the remaining material (in which the radioactive isotopes were sealed) does not cause any discomfort or harm to the patient. Permanent brachytherapy is a type of low-dose-rate brachytherapy. For temporary brachytherapy, tubes (catheters) or other carriers are used to deliver the radiation sources, and both the carriers and the radiation sources are removed after treatment. Temporary brachytherapy can be either low-dose-rate or high-dose-rate treatment. Brachytherapy may be used alone or in addition to external-beam radiation therapy to provide a “boost” of radiation to a tumor while sparing surrounding normal tissue.

In systemic radiation therapy, a patient may swallow or receive an injection of a radioactive substance, such as radioactive iodine or a radioactive substance bound to a monoclonal antibody. Radioactive iodine (131I) is a type of systemic radiation therapy commonly used to help treat cancer, such as thyroid cancer. Thyroid cells naturally take up radioactive iodine. For systemic radiation therapy for some other types of cancer, a monoclonal antibody may help target the radioactive substance to the right place. The antibody joined to the radioactive substance travels through the blood, locating and killing tumor cells. For example, the drug ibritumomab tiuxetan (Zevalin®) may be used for the treatment of certain types of B-cell non-Hodgkin lymphoma (NHL). The antibody part of this drug recognizes and binds to a protein found on the surface of B lymphocytes. The combination drug regimen of tositumomab and iodine 113I tositumomab (Bexxar®) may be used for the treatment of certain types of cancer, such as NHL. In this regimen, nonradioactive tositumomab antibodies may be given to patients first, followed by treatment with tositumomab antibodies that have 131I attached. Tositumomab may recognize and bind to the same protein on B lymphocytes as ibritumomab. The nonradioactive form of the antibody may help protect normal B lymphocytes from being damaged by radiation from 131I.

Some systemic radiation therapy drugs relieve pain from cancer that has spread to the bone (bone metastases). This is a type of palliative radiation therapy. The radioactive drugs samarium-153-lexidronam (Quadramet®) and strontium-89 chloride (Metastron®) are examples of radiopharmaceuticals may be used to treat pain from bone metastases.

Photodynamic therapy (PDT) is an anti-cancer treatment that may use a drug, called a photosensitizer or photosensitizing agent, and a particular type of light. When photosensitizers are exposed to a specific wavelength of light, they may produce a form of oxygen that kills nearby cells. A photosensitizer may be activated by light of a specific wavelength. This wavelength determines how far the light can travel into the body. Thus, photosensitizers and wavelengths of light may be used to treat different areas of the body with PDT.

In the first step of PDT for cancer treatment, a photosensitizing agent may be injected into the bloodstream. The agent may be absorbed by cells all over the body but may stay in cancer cells longer than it does in normal cells. Approximately 24 to 72 hours after injection, when most of the agent has left normal cells but remains in cancer cells, the tumor can be exposed to light. The photosensitizer in the tumor can absorb the light and produces an active form of oxygen that destroys nearby cancer cells. In addition to directly killing cancer cells, PDT may shrink or destroy tumors in two other ways. The photosensitizer can damage blood vessels in the tumor, thereby preventing the cancer from receiving necessary nutrients. PDT may also activate the immune system to attack the tumor cells.

The light used for PDT can come from a laser or other sources. Laser light can be directed through fiber optic cables (thin fibers that transmit light) to deliver light to areas inside the body. For example, a fiber optic cable can be inserted through an endoscope (a thin, lighted tube used to look at tissues inside the body) into the lungs or esophagus to treat cancer in these organs. Other light sources include light-emitting diodes (LEDs), which may be used for surface tumors, such as skin cancer. PDT is usually performed as an outpatient procedure. PDT may also be repeated and may be used with other therapies, such as surgery, radiation, or chemotherapy.

Extracorporeal photopheresis (ECP) is a type of PDT in which a machine may be used to collect the patient's blood cells. The patient's blood cells may be treated outside the body with a photosensitizing agent, exposed to light, and then returned to the patient. ECP may be used to help lessen the severity of skin symptoms of cutaneous T-cell lymphoma that has not responded to other therapies. ECP may be used to treat other blood cancers, and may also help reduce rejection after transplants.

Additionally, photosensitizing agent, such as porfimer sodium or Photofrin®, may be used in PDT to treat or relieve the symptoms of esophageal cancer and non-small cell lung cancer. Porfimer sodium may relieve symptoms of esophageal cancer when the cancer obstructs the esophagus or when the cancer cannot be satisfactorily treated with laser therapy alone. Porfimer sodium may be used to treat non-small cell lung cancer in patients for whom the usual treatments are not appropriate, and to relieve symptoms in patients with non-small cell lung cancer that obstructs the airways. Porfimer sodium may also be used for the treatment of precancerous lesions in patients with Barrett esophagus, a condition that can lead to esophageal cancer.

Laser therapy may use high-intensity light to treat cancer and other illnesses. Lasers can be used to shrink or destroy tumors or precancerous growths. Lasers are most commonly used to treat superficial cancers (cancers on the surface of the body or the lining of internal organs) such as basal cell skin cancer and the very early stages of some cancers, such as cervical, penile, vaginal, vulvar, and non-small cell lung cancer.

Lasers may also be used to relieve certain symptoms of cancer, such as bleeding or obstruction. For example, lasers can be used to shrink or destroy a tumor that is blocking a patient's trachea (windpipe) or esophagus. Lasers also can be used to remove colon polyps or tumors that are blocking the colon or stomach.

Laser therapy is often given through a flexible endoscope (a thin, lighted tube used to look at tissues inside the body). The endoscope is fitted with optical fibers (thin fibers that transmit light). It is inserted through an opening in the body, such as the mouth, nose, anus, or vagina. Laser light is then precisely aimed to cut or destroy a tumor.

Laser-induced interstitial thermotherapy (LITT), or interstitial laser photocoagulation, also uses lasers to treat some cancers. LITT is similar to a cancer treatment called hyperthermia, which uses heat to shrink tumors by damaging or killing cancer cells. During LITT, an optical fiber is inserted into a tumor. Laser light at the tip of the fiber raises the temperature of the tumor cells and damages or destroys them. LITT is sometimes used to shrink tumors in the liver.

Laser therapy can be used alone, but most often it is combined with other treatments, such as surgery, chemotherapy, or radiation therapy. In addition, lasers can seal nerve endings to reduce pain after surgery and seal lymph vessels to reduce swelling and limit the spread of tumor cells.

Lasers used to treat cancer may include carbon dioxide (CO₂) lasers, argon lasers, and neodymium:yttrium-aluminum-garnet (Nd:YAG) lasers. Each of these can shrink or destroy tumors and can be used with endoscopes. CO₂ and argon lasers can cut the skin's surface without going into deeper layers. Thus, they can be used to remove superficial cancers, such as skin cancer. In contrast, the Nd:YAG laser is more commonly applied through an endoscope to treat internal organs, such as the uterus, esophagus, and colon. Nd:YAG laser light can also travel through optical fibers into specific areas of the body during LITT. Argon lasers are often used to activate the drugs used in PDT.

Immunotherapy (sometimes called, biological therapy, biotherapy, biologic therapy, or biological response modifier (BRM) therapy) uses the body's immune system, either directly or indirectly, to fight cancer or to lessen the side effects that may be caused by some cancer treatments. Immunotherapies include interferons, interleukins, colony-stimulating factors, monoclonal antibodies, vaccines, immune cell-based therapy, gene therapy, and nonspecific immunomodulating agents.

Interferons (IFNs) are types of cytokines that occur naturally in the body. Interferon alpha, interferon beta, and interferon gamma are examples of interferons that may be used in cancer treatment.

Like interferons, interleukins (ILs) are cytokines that occur naturally in the body and can be made in the laboratory. Many interleukins have been identified for the treatment of cancer. For example, interleukin-2 (IL-2 or aldesleukin), interleukin 7, and interleukin 12 have may be used as an anti-cancer treatment. IL-2 may stimulate the growth and activity of many immune cells, such as lymphocytes, that can destroy cancer cells.

Colony-stimulating factors (CSFs) (sometimes called hematopoietic growth factors) may also be used for the treatment of cancer. Some examples of CSFs include, but are not limited to, G-CSF (filgrastim) and GM-CSF (sargramostim). CSFs may promote the division of bone marrow stem cells and their development into white blood cells, platelets, and red blood cells. Bone marrow is critical to the body's immune system because it is the source of all blood cells. Because anticancer drugs can damage the body's ability to make white blood cells, red blood cells, and platelets, stimulation of the immune system by CSFs may benefit patients undergoing other anti-cancer treatment, thus CSFs may be combined with other anti-cancer therapies, such as chemotherapy.

Another type of immunotherapy includes monoclonal antibodies (MOABs or MoABs). These antibodies may be produced by a single type of cell and may be specific for a particular antigen. To create MOABs, a human cancer cells may be injected into mice. In response, the mouse immune system can make antibodies against these cancer cells. The mouse plasma cells that produce antibodies may be isolated and fused with laboratory-grown cells to create “hybrid” cells called hybridomas. Hybridomas can indefinitely produce large quantities of these pure antibodies, or MOABs. MOABs may be used in cancer treatment in a number of ways. For instance, MOABs that react with specific types of cancer may enhance a patient's immune response to the cancer. MOABs can be programmed to act against cell growth factors, thus interfering with the growth of cancer cells.

MOABs may be linked to other anti-cancer therapies such as chemotherapeutics, radioisotopes (radioactive substances), other biological therapies, or other toxins. When the antibodies latch onto cancer cells, they deliver these anti-cancer therapies directly to the tumor, helping to destroy it. MOABs carrying radioisotopes may also prove useful in diagnosing cancer.

Cancer vaccines are another form of immunotherapy. Cancer vaccines may be designed to encourage the patient's immune system to recognize cancer cells. Cancer vaccines may be designed to treat existing cancers (therapeutic vaccines) or to prevent the development of cancer (prophylactic vaccines). Therapeutic vaccines may be injected in a person after cancer is diagnosed. These vaccines may stop the growth of existing tumors, prevent cancer from recurring, or eliminate cancer cells not killed by prior treatments. Cancer vaccines given when the tumor is small may be able to eradicate the cancer. On the other hand, prophylactic vaccines are given to healthy individuals before cancer develops. These vaccines are designed to stimulate the immune system to attack viruses that can cause cancer. By targeting these cancer-causing viruses, development of certain cancers may be prevented. For example, cervarix and gardasil are vaccines to treat human papilloma virus and may prevent cervical cancer. Therapeutic vaccines may be used to treat bladder cancer. Cancer vaccines can be used in combination with other anti-cancer therapies.

Immune cell-based therapy is also another form of immunotherapy. Adoptive cell transfer may include the transfer of immune cells such as dendritic cells, T cells (e.g., cytotoxic T cells), or natural killer (NK) cells to activate a cytotoxic response or attack cancer cells in a patient. Autologous immune cell-based therapy involves the transfer of a patient's own immune cells after expansion in vitro.

Gene therapy is another example of a biological therapy. Gene therapy may involve introducing genetic material into a person's cells to fight disease. Gene therapy methods may improve a patient's immune response to cancer. For example, a gene may be inserted into an immune cell to enhance its ability to recognize and attack cancer cells. In some embodiments, cancer cells may be injected with genes that cause the cancer cells to produce cytokines and stimulate the immune system.

In some instances, biological therapy includes nonspecific immunomodulating agents. Nonspecific immunomodulating agents are substances that stimulate or indirectly augment the immune system. Often, these agents target key immune system cells and may cause secondary responses such as increased production of cytokines and immunoglobulins. Two nonspecific immunomodulating agents used in cancer treatment are bacillus Calmette-Guerin (BCG) and levamisole. BCG may be used in the treatment of superficial bladder cancer following surgery. BCG may work by stimulating an inflammatory, and possibly an immune, response. A solution of BCG may be instilled in the bladder. Levamisole is sometimes used along with fluorouracil (5-FU) chemotherapy in the treatment of stage III (Dukes' C) colon cancer following surgery. Levamisole may act to restore depressed immune function.

Target sequences can be grouped so that information obtained about the set of target sequences in the group can be used to make or assist in making a clinically relevant judgment such as a diagnosis, prognosis, or treatment choice.

A patient report is also provided comprising a representation of measured expression levels of a plurality of target sequences in a biological sample from the patient, wherein the representation comprises expression levels of target sequences corresponding to any one, two, three, four, five, six, eight, ten, twenty, thirty or more of the target sequences corresponding to a target selected from Table 2 and/or Table 5, the subsets described herein, or a combination thereof. In some embodiments, the representation of the measured expression level(s) may take the form of a linear or nonlinear combination of expression levels of the target sequences of interest. The patient report may be provided in a machine (e.g., a computer) readable format and/or in a hard (paper) copy. The report can also include standard measurements of expression levels of said plurality of target sequences from one or more sets of patients with known disease status and/or outcome. The report can be used to inform the patient and/or treating physician of the expression levels of the expressed target sequences, the likely medical diagnosis and/or implications, and optionally may recommend a treatment modality for the patient.

Also provided are representations of the gene expression profiles useful for treating, diagnosing, prognosticating, and otherwise assessing disease. In some embodiments, these profile representations are reduced to a medium that can be automatically read by a machine such as computer readable media (magnetic, optical, and the like). The articles can also include instructions for assessing the gene expression profiles in such media. For example, the articles may comprise a readable storage form having computer instructions for comparing gene expression profiles of the portfolios of genes described above. The articles may also have gene expression profiles digitally recorded therein so that they may be compared with gene expression data from patient samples. In some instances, the profiles can be recorded in different representational format. A graphical recordation is one such format. Clustering algorithms can assist in the visualization of such data.

### II. Experimental

Below are examples of specific embodiments for carrying out the methods, compositions, systems and kits described herein. The examples are offered for illustrative purposes only, and are not intended to limit the scope of the present disclosure in any way.

Efforts have been made to ensure accuracy with respect to numbers used (e.g., amounts, temperatures, etc.), but some experimental error and deviation should, of course, be allowed for.

### EXAMPLES

### Example 1: Validation Study of Molecular Subtypes for Prognosis of Muscle-Invasive Bladder Cancer Using Genomic Classifiers with Long Non-Coding RNA Expression Profiling

Approximately 25% of patients present with muscle-invasive bladder cancer (MIBC). The recommended treatment option for MIBC is neoadjuvant cisplatin-based chemotherapy (NAC) followed by pelvic lymph node dissection and radical cystectomy (RC). Despite this aggressive treatment regimen, the 5-year overall survival (OS) is only approximately 55% from the time of surgery. Stratifying MIBC by molecular subtype has potential clinical value in terms of predicting both outcome and response to treatment, such as NAC or immunotherapy.

The significance of subtyping methods for the prognosis of muscle-invasive bladder cancer using long non-coding RNA (lncRNA) expression profiling was examined as follows.

### Patient Populations & Expression Data

For the present study, four MIBC patient cohorts were analyzed (see Table 1). 1) NAC cohort: A cohort of 223 MIBC patients from seven institutions who had received neoadjuvant/induction chemotherapy followed by radical cystectomy (RC) for cT2-4aN0-3M0 urothelial carcinoma of the bladder were compiled (Seiler R et al. (2017) Eur Urol 72:544-54). Whole transcriptome profiling had previously been performed on formalin-fixed, paraffin-embedded (FFPE), pre-treatment tissue samples from transurethral bladder tumor resection (TURBT) in a Clinical Laboratory Improvement Amendments (CLIA)-certified laboratory (GenomeDx Inc, San Diego, Calif.) (Erho N et al. (2013) PLoS One. 8:e66855). 2) TCGA cohort: RNA-seq data of 405 MIBC patients treated with RC in the absence of NAC was publicly available and previously analyzed by The Cancer Genome Atlas (TCGA) Research Network (Robertson A G et al. (2017) Cell 171:540-56 e25). 3) PCC cohort: A prospective commercial cohort (PCC) consisting of the de-identified and anonymized gene expression profiles of 255 MIBC patients from the clinical use of the Decipher Bladder TURBT test that were available in the Decipher GRID registry (NCT02609269). Pathological staging and clinical outcome data were not available for this cohort. 4) UTSW cohort: The UT Southwestern (UTSW) cohort consisting of 94 MIBC patients from the UT Southwestern Medical Center who underwent RC without neoadjuvant therapy. In this cohort, whole transcriptome profiling was performed on RC tissue samples. The NAC, PCC and UTSW cohorts were all profiled on the GeneChipl Human Exon 1.0 ST Array (Affymetrix).

TABLE 1
Clinicopathological characteristics of all patient cohorts

| | GC | | | | | | |
|---|---|---|---|---|---|---|---|
| | Training | | Testing | | Validation | | |
| | Cohort | | | | | | |
| | NAC | | TCGA | | UTSW | | PCC |
| N | 223 | | 405 | | 94 | | 255 |
| Tissue | TURBT | | RC | | RC | | TURBT |
| Expression | Array | | RNA-seq | | Array | | Array |
| Data | | | | | | | |
| Age—Median | 62 | (56-71) | 69 | (60-76) | 70 | (63-77) | NA |
| [IQR] | | | | | | | |
| Gender (%) | | | | | | | |
| Female | 69 | (31%) | 106 | (26%) | 16 | (17%) | NA |
| Male | 154 | (69%) | 299 | (74%) | 78 | (83%) | NA |
| Clinical lymph | | | | | | | |
| node stage (%) | | | | | | | |
| cN0 | 140 | (63%) | NA | | 94 | (100%) | NA |
| cN1-3 | 83 | (37%) | NA | | 0 | (0%) | NA |
| cNx | 0 | (0%) | NA | | 0 | (0%) | NA |
| Clinical tumor | | | | | | | |
| stage (%) | | | | | | | |
| cTis/Ta | 0 | (0%) | NA | | 4 | (4%) | NA |
| cT1 | 0 | (0%) | NA | | 10 | (11% | NA |
| cT2 | 90 | (40%) | NA | | 66 | (70%) | NA |
| cT3 | 90 | (40%) | NA | | 9 | (10%) | NA |
| cT4 | 43 | (20%) | NA | | 4 | (4%) | NA |
| cTx | 0 | (0%) | NA | | 1 | (1%) | NA |
| Pathological | | | | | | | |
| tumor stage | | | | | | | |
| (%) | | | | | | | |
| ypT0/Tis/Ta/T1 | 103 | (46%) | 0 | (0%) | 1 | (1%) | NA |
| ypT2 | 42 | (19%) | 122 | (30%) | 36 | (38%) | NA |
| ypT3 | 50 | (22%) | 193 | (48%) | 42 | (45%) | NA |
| ypT4 | 24 | (11%) | 57 | (14%) | 15 | (16%) | NA |
| ypTx | 4 | (2%) | 33 | (8%) | 0 | (0%) | NA |
| Pathological | | | | | | | |
| lymph node | | | | | | | |
| stage (%) | | | | | | | |
| ypN0 | 138 | (62%) | 235 | (58%) | 62 | (66%) | NA |
| ypN1-3 | 48 | (21%) | 129 | (32%) | 31 | (33%) | NA |
| ypNx | 37 | (17%) | 41 | (10%) | 1 | (1%) | NA |
| FGFR3+ eases | 36 | (16%) | 55 | (14%) | 10 | (11%) | 24 (11%) |

| | | | | |
|---|---|---|---|---|
| by GC (%) | | | | |
| of which n died | 2 | 9 | 1 | NA |
| during follow | | | | |
| up: | | | | |

Unsupervised Clustering Using lncRNAs

For unsupervised clustering analysis the normalized gene expression data for n=223 samples (NAC cohort) was pre-processed by multi-analysis distance sampling (R package MADS) to identify highly-variant lncRNA genes. The expression clustering analysis was done by a consensus partitioning around medoids (PAM) approach, using Spearman correlations, 10,000 iterations with a 0.95 random fraction of lncRNAs in each iteration (R package ConsensusClusterPlus). This process was repeated with log-transformed, RNA-seq gene expression data (TCGA cohort) for n=405 samples, to see whether clustering of our de nowo selected lncRNA genes would identify lncRNA clusters that were similar to those identified by the TCGA analysis (Robertson A G et al. (2017) Cell 171:540-56 e25). Concordance of this cluster solution was determined with the published lncRNA cluster solution using Cohen's kappa statistic.

Classification of Tumors Among Molecular mRNA Subtypes

A classifier was generated that was based on the published TCGA 2017 mRNA subtypes (Robertson A G et al. (2017) Cell 171:540-56 e25), to classify tumors from the NAC, PCC and UTSW cohorts into basal/squamous, luminal, luminal-infiltrated, luminal-papillary, and neuronal mRNA subtypes. An additional category was introduced, ‘unknown’, to provide a bin for tumors that did not fit the aforementioned subtyping structure. Furthermore, the recently released consensus molecular classification by The Bladder Cancer Molecular Taxonomy Group was applied to classify tumors from all four cohorts into six consensus mRNA subtypes: basal/squamous, luminal-papillary, luminal non-specified, luminal unstable, stroma-rich, and neuroendocrine-like (Kamoun A et al. (2018) BioRxiv Preprint).

Regulon Analysis of lncRNA Clusters

Regulon analysis involves calculations that transform a cohort's gene expression data into a functional readout that can inform on biological state (Campbell T M et al. (2016) Carcinogenesis. 37:741-50 and Castro M A et al. (2016) Nat Genet. 48:12-21). An initial step reconstructs regulatory units, each of which consists of a regulator, i.e. a gene whose product induces and/or represses a set of target genes, which we call a ‘regulon’. A second step calculates the activity profile of a regulon across a cohort. As demonstrated for breast cancer (Campbell T M et al. (2016) Carcinogenesis. 37:741-50), and in the TCGA MIBC study (Robertson A G et al. (2017) Cell 171:540-56 e25), subsequent steps may use activity profiles as a molecular covariate to segregate clinical subtypes. In the studies disclosed herein, regulon activity profiles for both FGFR3 and SHH segregated FGFR3 and TP53 mutations, and the LPL-C3 tumors.

R package RTN v2.7.1 was used to calculate a transcriptional regulatory network from RSEM RNA-seq data for the TCGA-BLCA discovery cohort, as in Robertson et al ((2017) Cell 171:540-56 e25). A set of 26 regulators were used: the 23 from TCGA work (AR, EGFR, ERBB2, ERBB3, ESR, ESR2, FGFRI, FGFR3, FOXA1, FOAMJ, GATA3, GATA6, HIF1A, KLF4, PGR, PPARG, RARA, RARB, RARG, RXRA, RXRB, STAT3, and TP63), with RB1, SHH and TPS3 added. For calculating regulon activity profiles across a cohort, a regulon was required to have at least 15 positive and 15 negative targets. Regulon target genes from the discovery cohort were used to calculate regulon activities in the NAC validation cohort. For each regulon, enrichment tests (Fisher's Exact Tests) were performed to identify whether lncRNA clusters were enriched with samples of high or low regulon activity. RTNsurvival v1.6.0 and TCGA-BLCA mutation data was used to generate oncoprint-like diagrams that showed, for the TCGA cohort, how regulon activity segregated TP53 and FGFR3 mutations, and LPL-C3 and LPL-Other samples (Robertson A G et al. (2017) Cell 171:540-56 e25).

### Gene Expression Analysis

Heatmaps and boxplots were created to visualize differences between tumors from lncRNA and mRNA subtypes, in the expression of individual genes, gene signatures (Sjodahl G et al. (2012) Clin Cancer Res. 18:3377-86), and hallmark gene sets (from the molecular signatures database hallmark gene set collection, MSigDB (Liberzon A et al. (2015) Cell Syst. 1:417-25)). Hedgehog signaling activity was quantified by a signature based on target genes (SHH, BMP4, BAP5, ID1, ID2, ID3, ID4) as mentioned by Shin et al (2014) Cancer Cell. 26:521-33. Sample purity was calculated by the ABSOLUTE and ESTIMATE algorithms for the TCGA and NAC cohort, respectively (Carter S L et al. (2012) Nat Biotechnol. 30:413-21 and Yoshihara K et al. (2013) Nat Commun. 2013; 4:2612). Median fold changes (FC) and p-values (using two-sided Wilcoxon rank-sum tests) were calculated for differential gene expression analyses.

### Statistical Analyses

Statistical analyses were performed using R statistical software (R Foundation for Statistical Computing, Vienna, Austria). In the NAC and TCGA cohorts, patient and tumor characteristics were compared between subgroups by Fisher's exact tests and two-sided Wilcoxon rank-sum tests. The primary endpoint for the survival analysis was overall survival (OS). OS was calculated as the date of the most recent TURBT (NAC, PCC cohort) or RC (TCGA, UTSW cohort) till date of death from any cause. Patients who were lost to follow-up were censored at the date of last contact. The Kaplan-Meier method was used to estimate the statistical significance of differences between survival curves for patients of different molecular subtypes, using the log-rank test. After checking the proportional hazard assumption based on Schoenfeld residuals, multi-variate Cox proportional hazard models were used to demonstrate the relationship of the genomic classifier's predicted subtype with OS, adjusting for clinical variables, including age sex and stage.

### Discovery and Validation of a Genomic Classifier

The NAC cohort was used to train a genomic classifier (GC) to predict luminal-papillary MIBC patients that had favorable prognosis (OS), as identified by the lncRNA clustering (LPL-C3). To make the model applicable to several platforms, genes were selected that were present in both the Illumina HiSeq platform (TCGA cohort) and Affymetrix Human Exon 1.0 ST Array (NAC, PCC, UTSW cohorts) as the initial gene list (25,942 genes). Using this gene list, the selection of genes for the GC was based on overlap of gene sets that were created by differential gene expression analyses (median FC<−0.06 or >0.1, p<0.001), in which lncRNA clusters were compared with mRNA subtypes. This resulted in a list of 69 candidate genes. The final gene set included 65 genes after removing highly abundant mitochondrial transcripts (seven genes) and adding three genes enriched in LPL-C3, determined from heatmaps generated in the study (SHH, BMP5 and FGFR3) (see Table 2). Next, a 10-fold cross-validated, ridge-penalized logistic regression model, consisting of 36 coefficients, was trained to predict LPL-C3 MIBC (R package glmnet). This model was applied to RNA-seq data (TCGA) using quantile normalization. For the 65 genes, expression values from RNA-seq were normalized by quantile-quantile matching with the expression values in the training cohort (NAC) as implemented in R package preprocessCore. The R package OptimalCutpoints were used to select the optimal probability threshold (Pt), corresponding to the maximal specificity for identifying LPL-C3 MIBC patients in both NAC and TCGA cohorts. Finally, a probability threshold (Pt) of 0.43 was selected, corresponding to a 98-68% specificity-sensitivity combination in the NAC cohort and a 96-55% specificity-sensitivity combination in TCGA cohort. After training and testing of the GC in NAC and TCGA cohorts, the classifier was locked for further independent external validation in the PCC and UTSW cohorts.

TABLE 2
list of 65 gene features for the genomic classifier.

AC017060.1
AC019117.1
AC019117.2
ACOX1
ADAM10
ADIRF
AEBP1
AF038458.3
AGR2
AQP3
ATP8B1
BCAS1
BEX4
BHLHE41
BMP5
BTG2
CAT
CHI3L1
COL1A1
COL1A2
COL3A1
COL6A1
COL6A3
CTD-2340E1.2
CTD-2626G11.2
DHRS2
FGFR3
FRY
GPX2
GREM1
HMGCS2
HPGD
MECOM
PDE10A
PGAP1
PHGR1
PLCD3
POF1B
PPDPF
PTPN13
RNF128
RP11-172F10.1
RP11-473M20.16
RP11-488L18.10
RP11-58K22.1
RP11-706O15.3
SEMA5A
SEMA6A
SFRP4
SHH
SLC14A1
SLITRK6
SNURF
SORL1
SULF1
TBX3
TMPRSS4
TOX3
UGT1A1
UGT1A3
UGT1A5
UGT1A8
UGT1A9
ZNF626
ZNF737

LncRNA Expression Profiling Subdivides the Luminal-Papillary mRNA Subtype

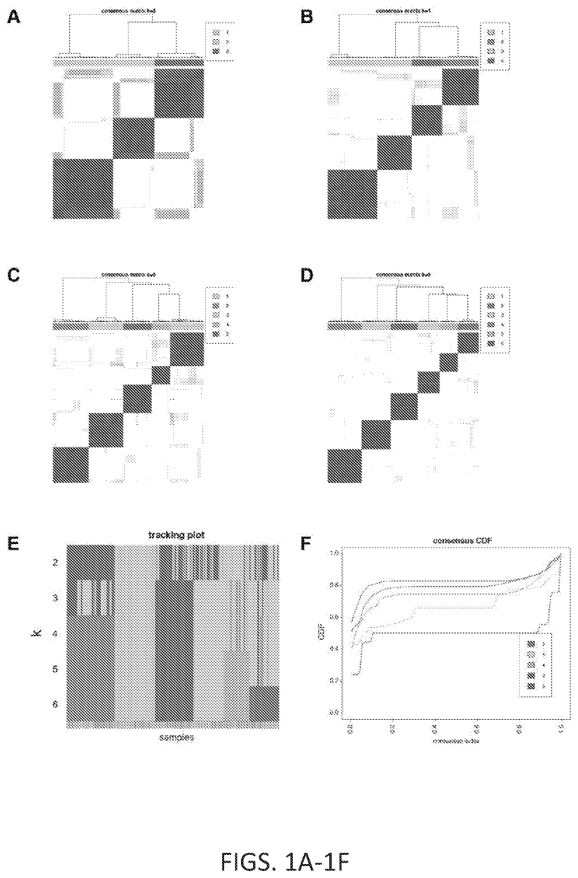

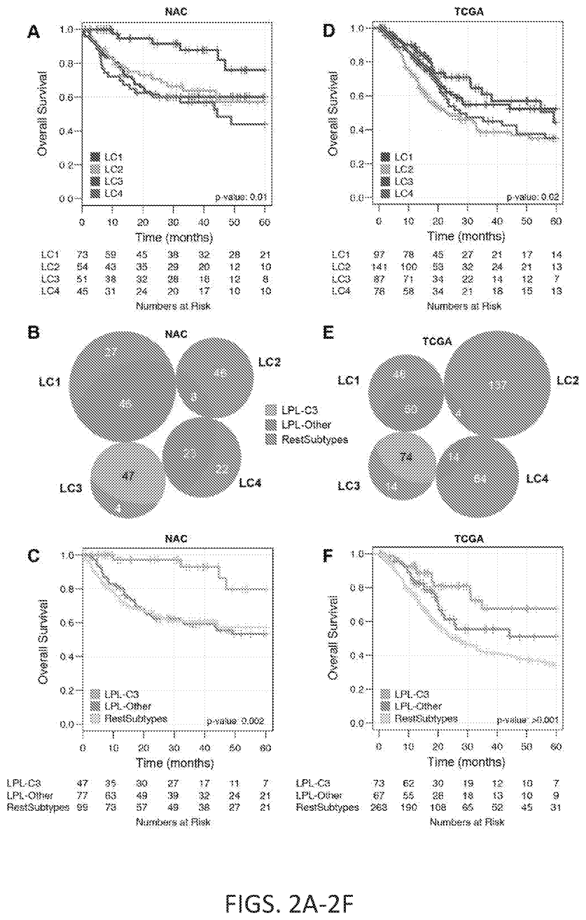

To explore the lncRNA expression landscape of MIBC, a microarray-based cohort of 223 bladder cancer TURBT samples treated with NAC and RC (NAC cohort) was downloaded. Unsupervised consensus clustering of 750 of the most highly-variant lncRNAs resulted in a robust four-cluster consensus solution (see FIG. 1). Survival analysis of the lncRNA-based consensus clusters (LC1-4) revealed that LC3 had significantly better prognosis than clusters LC1, LC2 and LC4 (p=0.01) (see FIG. 2A).

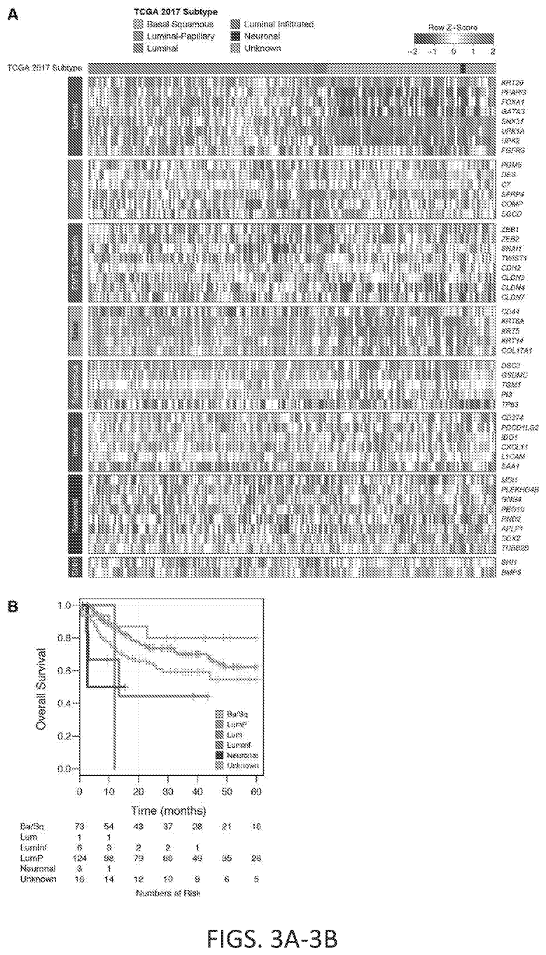

To assign the tumors in the NAC cohort to TCGA 2017 mRNA subtypes (luminal papillary, luminal, luminal infiltrated, basal squamous and neuronal), the single-sample classifier described above was applied, which revealed that these tumors were enriched for basal/squamous (33%) and luminal-papillary (54%) subtypes (see FIG. 3A). Survival analysis showed that patients with luminal-papillary tumors had better outcomes than the other subtypes (see FIG. 3B).

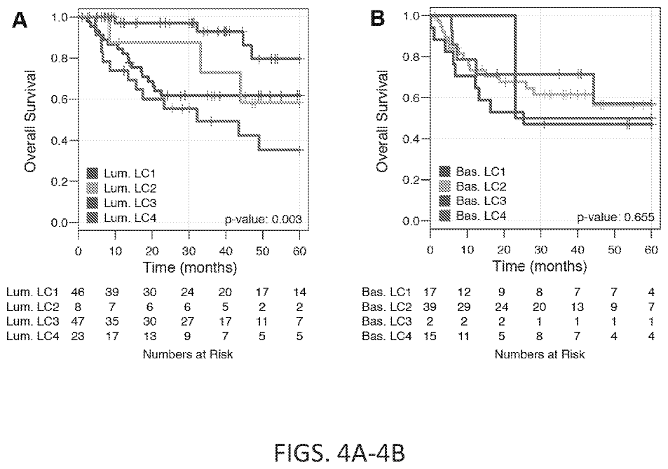

Comparing the lncRNA four-cluster solution and the classifier assigned TCGA subtypes, LC2 was strongly enriched (72%, 39/54) for tumors of the basal/squamous subtype, whereas LC1, LC3 and LC4 contained only 23%, 4% and 33% basal/squamous tumors, respectively (p<0.001). Conversely, luminal-papillary tumors were enriched in the LC3 (92%, 47/51) but were also present in the LC1 (63%) and LC4 (51%) clusters (p<0.001) (see FIG. 2B). Considering only the luminal-papillary subtype (n=124), patients in LC3 (38%) had favorable outcomes compared to other luminal-papillary tumors (p=0.003; see FIG. 2C and FIG. 4A), whereas stratifying the basal-squamous subtype by lncRNA clusters did not reveal differences in outcome (p=0.66; see FIG. 4B). Given the enrichment of luminal-papillary tumors in LC3, this group of patients was named ‘Luminal-Papillary LncRNA Cluster 3 (LPL-C3)’, and the other luminal-papillary tumors as ‘LPL-Other’.

Next, the consensus clustering in the TCGA cohort (n=405) was repeated using the lncRNAs that were consistent between the array and RNA-seq platforms (739/750). This resulted in a four-cluster consensus solution that was substantially concordant with the published TCGA lncRNA results (Robertson A G et al. (2017) Cell 171:540-56 e25) (k=0.77, p<0.001, see Table 3). As in the NAC cohort, a distinct lncRNA cluster (LC3) enriched in luminal-papillary tumors (74/88 patients, p<0.001) with favorable prognosis (p=0.0²²) was identified (see FIGS. 2D-F and Table 4).

TABLE 3
Concordance of our generated IncRNA clusters and the IncRNA
clusters from the TCGA 2017 publication, in the TCGA cohort.

| | | LucRNA clusters (739 | | | |
|---|---|---|---|---|---|
| | | IncRNAs) | | | |
| | | 1 | 2 | 3 | 4 |
| TCGA 2017 | 2 | 82 | 2 | 14 | 3 |
| LncRNA | 4 | 5 | 132 | 2 | 9 |
| clusters | | | | | |
| | 3 | 7 | 3 | 62 | 4 |
| | 1 | 4 | 4 | 10 | 62 |

Cohen's Kappa 0.774 p<0.001

TABLE 4
Intersection of the lncRNA-based consensus clusters and the
TCGA2017 mRNA subtypes for the NAC and TCGA cohorts.

| | NAC | | | | TCGA | | | |
|---|---|---|---|---|---|---|---|---|
| | LC1 | LC2 | LC3 | LC4 | LC1 | LC2 | LC3 | LC4 |
| Basal_Sq | 17 | 39 | 2 | 15 | 1 | 115 | 1 | 24 |
| Luminal | 0 | 0 | 1 | 0 | 19 | 0 | 3 | 3 |
| Luminal_Inf | 0 | 2 | 1 | 3 | 19 | 13 | 9 | 36 |
| Lum-pap | 46 | 8 | 47 | 21 | 50 | 4 | 74 | 14 |
| Neuronal | 0 | 1 | 0 | 2 | 9 | 9 | 1 | 1 |
| Unknown | 10 | 4 | 0 | 2 | na | na | na | na |
| Total | 73 | 54 | 51 | 45 | 98 | 141 | 88 | 78 |

The Biological Characteristics of LPL-C3 Tumors are Consistent with Less Aggressive Disease

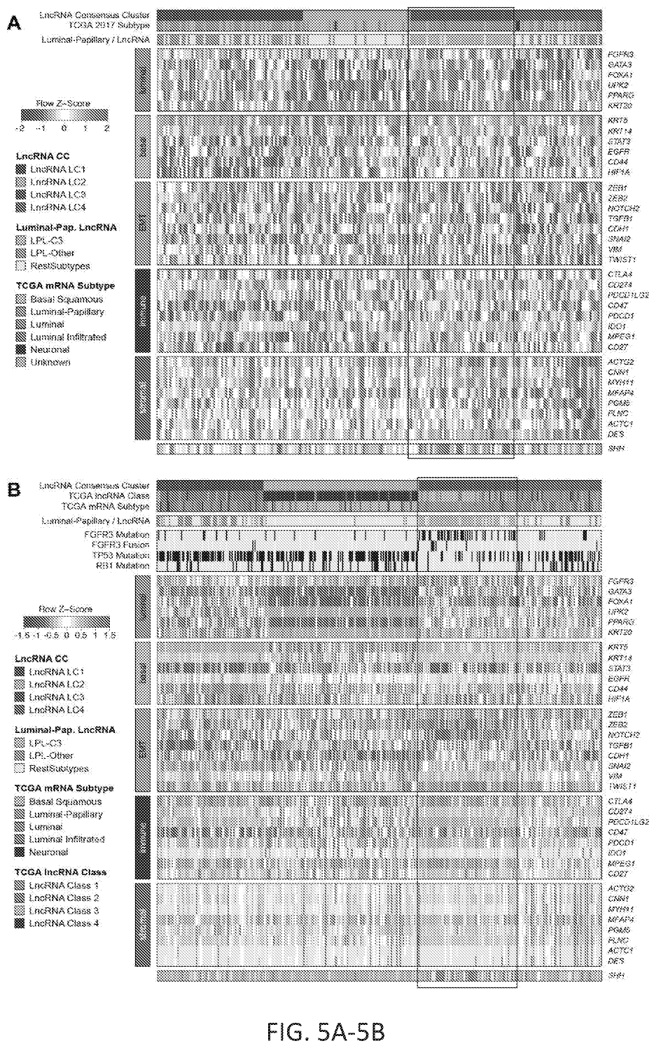

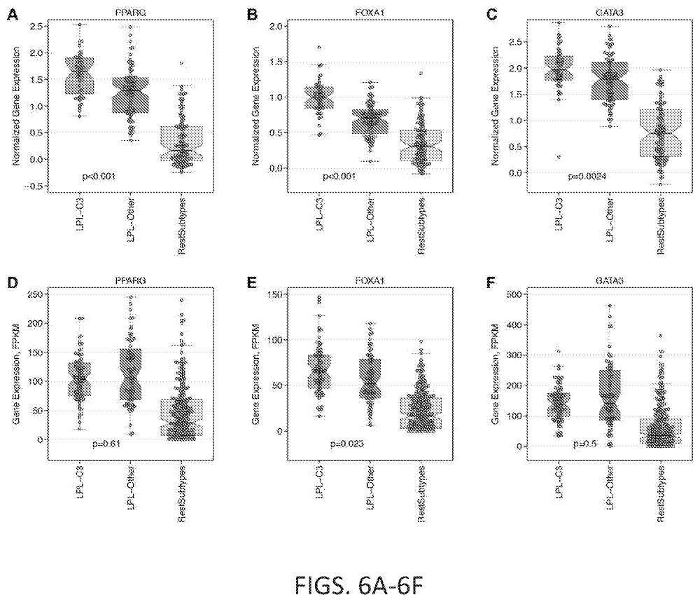

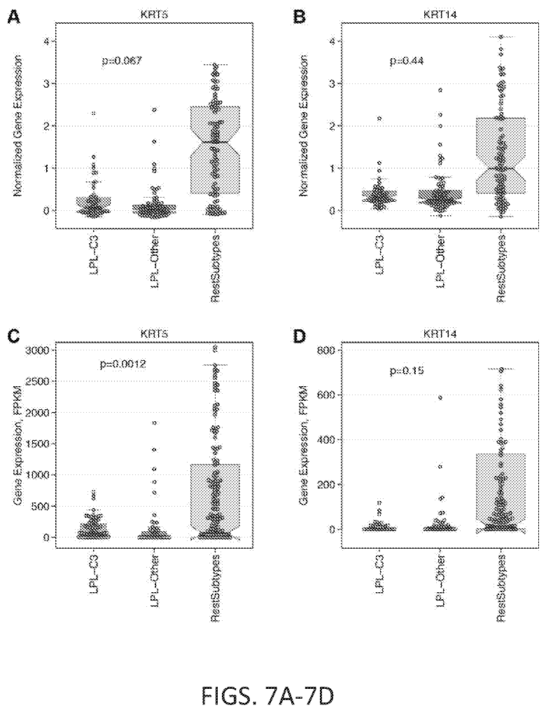

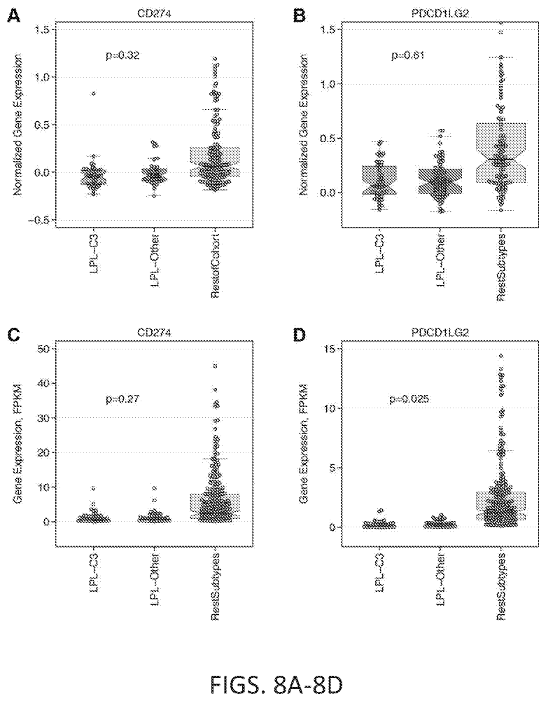

To investigate the biological differences between the LPL-C3 and LPL-Other tumors, a heatmap of genes associated with MIBC subtypes was generated for both the NAC and TCGA cohorts (see FIGS. 5A and 5B). Many luminal markers (i.e. PPARG, FOAfA, and GATA3) were expressed at significantly higher levels in LPL-C3 than in LPL-Other tumors (see FIGS. 6A-6C). These patterns were less evident in the TCGA cohort, with only FOIA1 showing significantly increased expression (p=0.023) (see FIGS. 6D-6F). In both cohorts, all luminal-papillary tumors showed downregulation of basal (i.e. KRT5′6, KRT14) (see FIGS. 5A, 5B, and FIG. 7) and immune-associated genes (i.e. CD274, PDCDILG2) (see FIGS. 5A, 5B, and FIG. 8).

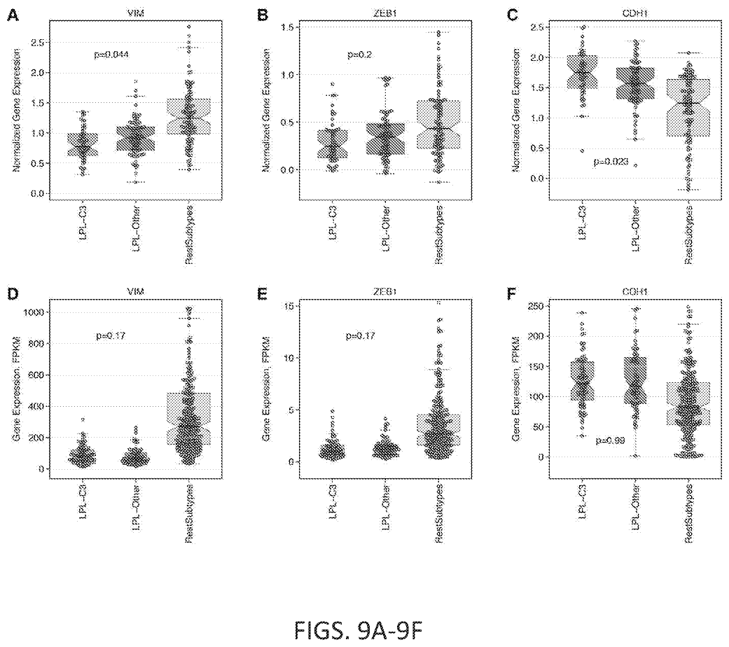

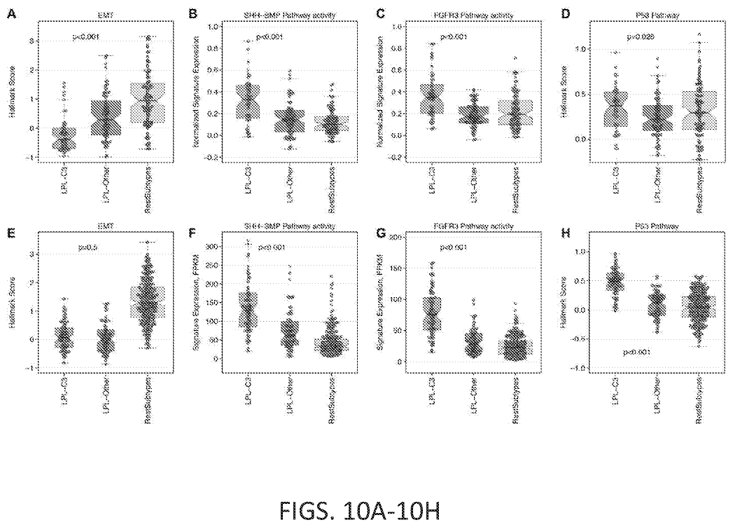

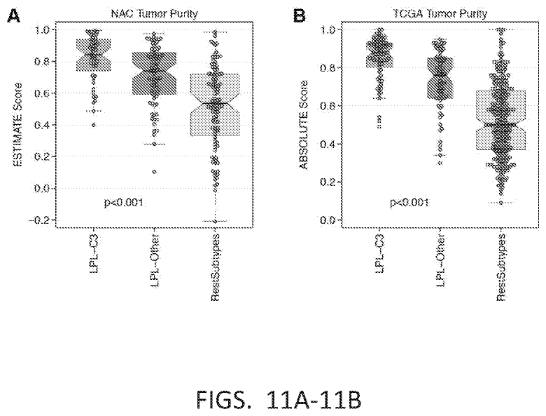

Significant differences in expression of genes associated with epidermal-to-mesenchymal transition (EMT) were observed for LPL-C3 versus LPL-Other tumors in the NAC cohort (see FIGS. 9A-9C). For example, VIM and ZEB1 were less abundant and CDH1 was more abundant in LPL-C3, indicating lower EMT activity in these tumors. Hallmark EMT signature scores were also significantly lower among the LPL-C3 tumors in the NAC cohort (see FIG. 10A). However, in the TCGA cohort EMT activity differences between LPL-C3 and LPL-Other tumors were not significant (p=0.5), although both luminal-papillary subsets showed low levels of both EMT gene expression and EMT hallmark scores (see FIG. 10E and FIGS. 9D-9F). Moreover, LPL-C3 tumors had the highest purity in both cohorts (see FIG. 11), precluding fibroblast infiltration as a possible cause of lower EMT hallmark scores.

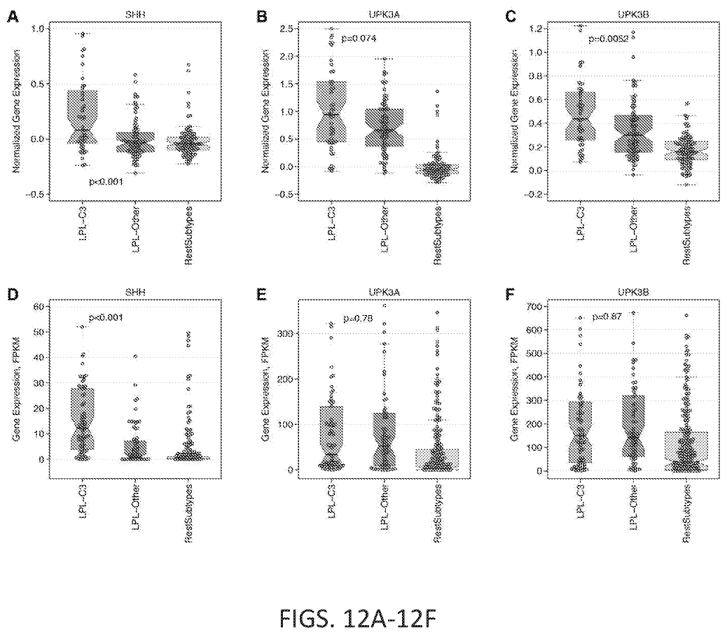

Higher expression of SHH and genes associated with urothelial differentiation (i.e. UPK3A, UPK3B) are features of luminal-papillary tumors (Robertson A G et al. (2017) Cell 171:540-56 e25 and Shin K et al. (2014) Cancer Cell. 26:521-33). In both cohorts, LPL-C3 tumors had higher expression of SHH (see FIG. 12) and SHH-BMP pathway activity signature scores (see FIGS. 10B and 10F).

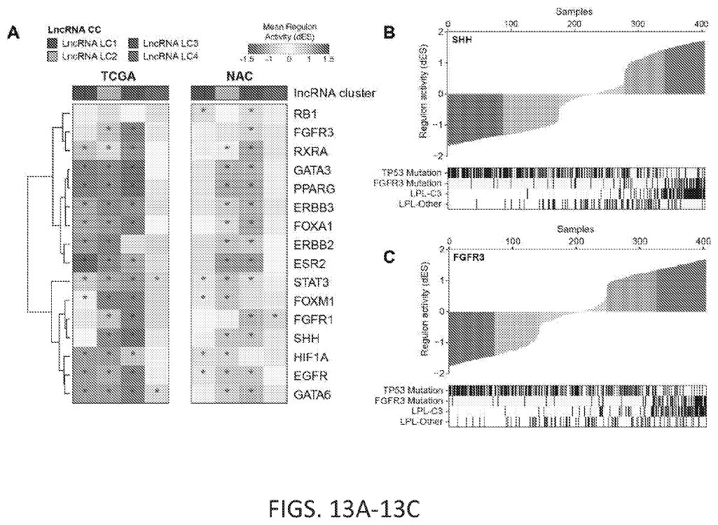

Next, regulon activities were used to further explore the differences in biology between the LPL-C3 tumors, the LPL-Other tumors and the rest of the cohort (Robertson A G et al. (2017) Cell 171:540-56 e25 and Castro M A et al. (2016) Nat Genet. 48:12-21), using the TCGA cohort for discovery and the NAC cohort for validation. Regulon analysis returns a profile of the activity of a transcription factor (or similar regulator) across a cohort (see Methods described above). Mean regulon activities for LC2 and LC3 subtypes were largely consistent between cohorts, though only weakly for LC1 (see FIG. 13A). Activated SHH and FGFR3 regulon activity was associated with LC3 (LPL-C3) tumors and enriched with FGFR3 mutations (see FIGS. 13B and 13C), consistent with the results of the gene expression analysis.

LPL-C3 Tunsors are Enriched for FGFR3 Alterations and have Wild-Type p53 Activity

A panel of 59 genes with mutation status reported in the TCGA cohort (Robertson A G et al. (2017) Cell 171:540-56 e25) was investigated as follows. After adjusting for false discovery rate (FDR), FGFR3, TP53 and RB1 were retrained, rates of mutation differed (p<0.05) between LPL-C3 and the rest of the cohort (see FIG. 5B and Table 5).

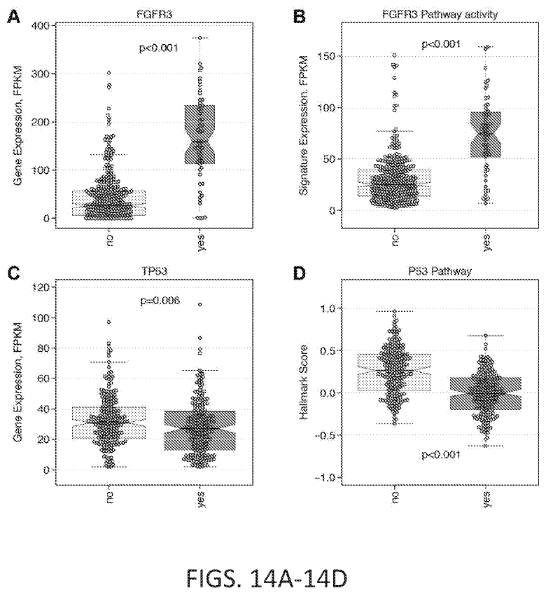

In the LPL-C3 tumors the enrichment for FGFR3-mutations (33/74 cases, p<0.001) correlated with both increased FGFR3 gene expression and signaling activity (see FIGS. 14A and 14B). These tumors were also enriched for FGFR3 fusions (6/74, p=0.02; see FIG. 5B), which was the only significant fusion event identified when comparing LPL-C3 and the rest of the cohort (see Table 6). Tumors with strongly activated FGFR3 regulon activity were likewise enriched in FGFR3 mutations, supporting this observation (FIG. 13C). Although FGFR3 mutation status was not available for the NAC cohort, both the FGFR3 gene expression and gene signature activity were significantly higher in the LPL-C3 tumors (p<0.001) (see FIG. 10C).

TABLE 5
Mutation status enrichment for LPL-C3 vs other in TCGA cohort.

| gene | p.value | FDR.q | gene | p.value | FDR.q |
|---|---|---|---|---|---|
| FGFR3 | 1.55E−13 | 9.16E−12 | CDKN2A | 0.44204953 | 0.82863808 |
| TP53 | 4.63E−10 | 1.37E−08 | TMCO4 | 0.46554914 | 0.82863808 |
| RB1 | 4.95E−06 | 9.74E−05 | KMT2D | 0.47605697 | 0.82863808 |
| CDKN1A | 0.00653568 | 0.07836488 | MBD1 | 0.47752025 | 0.82863808 |
| KMT2A | 0.00745818 | 0.07836488 | METTL3 | 0.52756469 | 0.86922348 |
| SSH3 | 0.00796931 | 0.07836488 | PIK3CA | 0.53745983 | 0.86922348 |
| STAG2 | 0.01389698 | 0.11713165 | CUL1 | 0.54654012 | 0.86922348 |
| SF3B1 | 0.01962343 | 0.14472278 | PSIP1 | 0.55983885 | 0.86922348 |
| ERCC2 | 0.02704477 | 0.1703768 | ERBB3 | 0.67273712 | 0.96458231 |
| KDM6A | 0.02887742 | 0.1703768 | CREBBP | 0.69405567 | 0.96458231 |
| TSC1 | 0.06340864 | 0.34010089 | HES1 | 0.69715906 | 0.96458231 |
| NFE2L2 | 0.09715199 | 0.45178587 | SF1 | 0.69715906 | 0.96458231 |
| KRAS | 0.09954604 | 0.45178587 | GNA13 | 0.70300066 | 0.96458231 |
| SPTAN1 | 0.1103669 | 0.45262303 | KMT2C | 0.74056131 | 0.99302539 |
| HRAS | 0.13184331 | 0.45262303 | KANSL1 | 0.78488196 | 1 |
| ARID1A | 0.13733428 | 0.45262303 | ZFP36L1 | 0.79996261 | 1 |
| HIST1H3B | 0.13808838 | 0.45262303 | ERBB2 | 0.84452857 | 1 |
| PTEN | 0.13808838 | 0.45262303 | ASXL2 | 1 | 1 |
| NRAS | 0.16448743 | 0.51077675 | ATM | 1 | 1 |
| NUP93 | 0.21553838 | 0.61800065 | C3orf70 | 1 | 1 |
| ACTB | 0.21996633 | 0.61800065 | CASP8 | 1 | 1 |
| RXRA | 0.27757769 | 0.7041601 | ELF3 | 1 | 1 |
| PARD3 | 0.28171697 | 0.7041601 | FAT1 | 1 | 1 |
| EP300 | 0.28643801 | 0.7041601 | FBXW7 | 1 | 1 |
| ZNF773 | 0.30196323 | 0.71263322 | RBM10 | 1 | 1 |
| MB21D2 | 0.3585823 | 0.76311301 | RHOB | 1 | 1 |
| TAF11 | 0.36045201 | 0.76311301 | SPN | 1 | 1 |
| RHOA | 0.36215533 | 0.76311301 | USP28 | 1 | 1 |
| ASXL1 | 0.41234587 | 0.81094688 | ZBTB7B | 1 | 1 |
| KLF5 | 0.41234587 | 0.81094688 | | | |

TABLE 6
Fusion event enrichment for LPL-C3 vs other in TCGA cohort.

| gene | p.value | FDR.q |
|---|---|---|
| FGFR3 | 0.003508 | 0.021049 |
| PTPN13 | 0.5897 | 1 |
| PPARG | 0.697159 | 1 |
| ASIP | 1 | 1 |
| RHOA | 1 | 1 |
| TNFRSF21 | 1 | 1 |

To examine if TP53 mutation correlated with impaired p53 activity, expression of the p53 pathway hallmark scores between TP53 mutated and wild-type patients were compared within the TCGA-cohort (see FIGS. 14C and 14D). The LPL-C3 tumors, which were depleted for 7P53 mutations, showed the highest p53 hallmark scores, which suggested functional p53 activity (see FIG. 5B and FIG. 10H). Consistent with this, samples with high SHH and FGFR3 regulon activities were depleted in TP53 mutation (see FIGS. 13B and 13C). Unfortunately, the TP53 regulon had insufficient (<15) positive and negative targets and was therefore too small to support activity calculations. The TP53 regulon was therefore excluded from the analysis. Although 77P53 mutation status was not available for the NAC cohort, the LPL-C3 tumors had higher p53 hallmark scores, suggesting these tumors may also be depleted for TP53 mutations (see FIG. 10G).

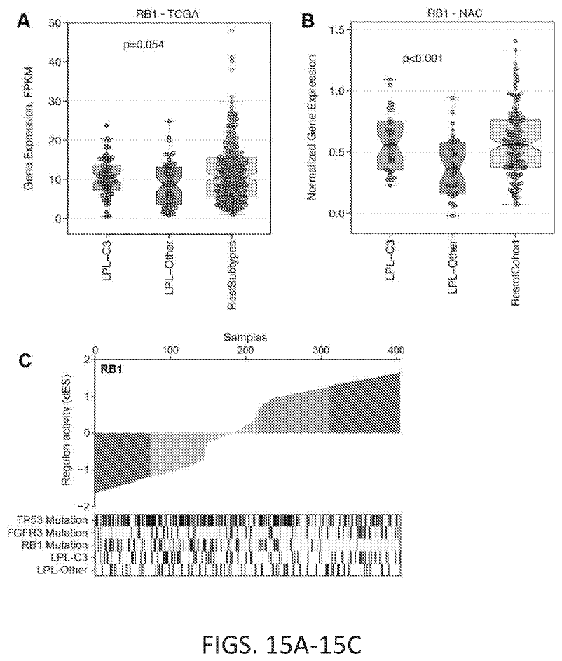

Although LPL-C3 tumors from the TCGA cohort were depleted for RB1 mutations, RB1 gene expression differed only non-significantly between LPL subgroups (p=0.0⁵⁴) (see FIG. 5B and FIG. 15A). In contrast, LPL-C3 tumors from the NAC cohort had significantly higher expression of RB1 (p=5.5×10⁻⁴) (see FIG. 5A and FIG. 15B). In contrast to SHH and FGFR3 regulon activities, tumors with higher RB1 regulon activity showed only weak depletion for TP53 mutations in the TCGA cohort (see FIG. 15C).

All gene and pathway activities of LPL-C3 tumors suggested that these tumors should be less clinically aggressive. Therefore, the clinical features of luminal-papillary patients in the NAC cohort were compared and higher rates of organ-confined disease were identified, including significantly lower pT-stage (p=0.047) and fewer lymph node metastases (p=0.0016) for LPL-C3 tumors (see Table 7). Similar observations were seen in the TCGA cohort, with lower ypT-stage (p=0.0043) and fewer lymph node metastases in LPL-C3 patients (p=0.002). In the NAC and TCGA cohorts, the median age of patients with LPL-C3 tumors was significantly lower (median age 58 vs. 63 years and 61 vs. 70 years, respectively; p<0.01).

TABLE 7
Clinicopathological characteristics of luminal-papillary MIBC patients from the NAC
and TCGA cohorts

| | NAC | | | | | TCGA | | | | |
|---|---|---|---|---|---|---|---|---|---|---|
| | Luminal-papillary subset | | | | | | | | | |
| | LPL-C3 | | LPL-Other | | | LPL-C3 | | LPL-Other | | |
| | (n = 47) | | (n = 77) | | p-value | (n = 74) | | (n = 68) | | p-value |
| Age - Median | 58 | (51-65) | 63 | (58-72) | 0.00098 | 61 | (54-71) | 70 | (64-77) | 0.0034 |
| [IQR] | | | | | | | | | | |
| Gender (%) | | | | | 1.00 | | | | | 1.00 |
| Female | 12 | (26%) | 19 | (25%) | | 16 | (22%) | 14 | (21%) | |
| Male | 35 | (74%) | 58 | (75%) | | 58 | (78%) | 54 | (79%) | |
| Clinical lymph | | | | | 0.00075 | | | | | NA |
| node stage (%) | | | | | | | | | | |
| cN0 | 36 | (77%) | 35 | (45%) | | NA | | NA | | |
| cN1-3 | 11 | (23%) | 42 | (55%) | | NA | | NA | | |
| cNx | 0 | (0%) | 0 | (0%) | | NA | | NA | | |
| Clinical tumor | | | | | 0.64 | | | | | NA |
| stage (%) | | | | | | | | | | |
| Tis/Ta/T1 | | | | | | | | | | |
| cT2 | 23 | (49%) | 33 | (43%) | | NA | | NA | | |
| cT3 | 18 | (38%) | 29 | (38%) | | NA | | NA | | |
| cT4 | 6 | (13%) | 15 | (19%) | | NA | | NA | | |
| Pathological | | | | | 0.047 | | | | | 0.0043 |
| Tumor Stage | | | | | | | | | | |
| (%) | | | | | | | | | | |
| ypT0/Tis/Ta/T1 | 28 | (59%) | 32 | (42%) | | 0 | | 0 | | |
| ypT2 | 13 | (28%) | 17 | (22%) | | 44 | (59%) | 21 | (31%) | |
| ypT3 | 5 | (11%) | 20 | (26%) | | 16 | (22%) | 28 | (41%) | |
| ypT4 | 1 | (2%) | 7 | (9%) | | 7 | (9.5%) | 6 | (9%) | |
| ypTx | 0 | (0%) | 1 | (1%) | | 7 | (95%) | 13 | (19%) | |
| Pathological | | | | | 0.0016 | | | | | 0.0020 |
| lymph node | | | | | | | | | | |
| stage (%) | | | | | | | | | | |
| yN0 | 39 | (83%) | 35 | (45%) | | 61 | (82%) | 36 | (53%) | |
| yN1-3 | 5 | (11%) | 24 | (31%) | | 6 | (8%) | 17 | (25%) | |
| yNx | 3 | (6) | 18 | (23%) | | 7 | (9%) | 15 | (22%) | |

Development of a Single-Sample Classifier to Identify Luminal-Papillary MIBC Patients with Good Prognosis

To provide utility as a prognostic model, a single-sample genomic classifier (GC) to identify the good-prognosis luminal tumors with activated FGFR3 (FGFR3+) was developed as follows. To be classified as FGFR3+, the tumor must also show enhanced SHH activity, higher p53 pathway activity and lower EMT, consistent with the data shown above.

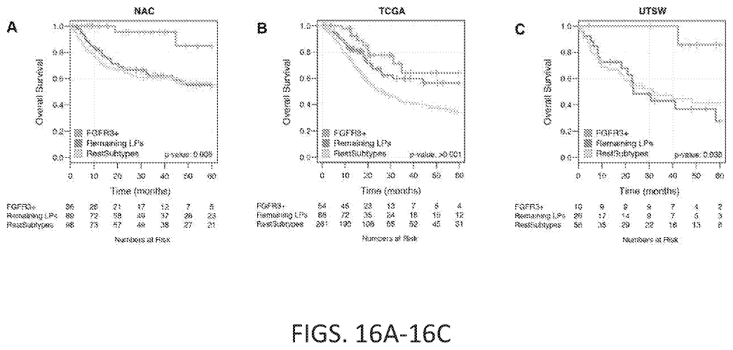

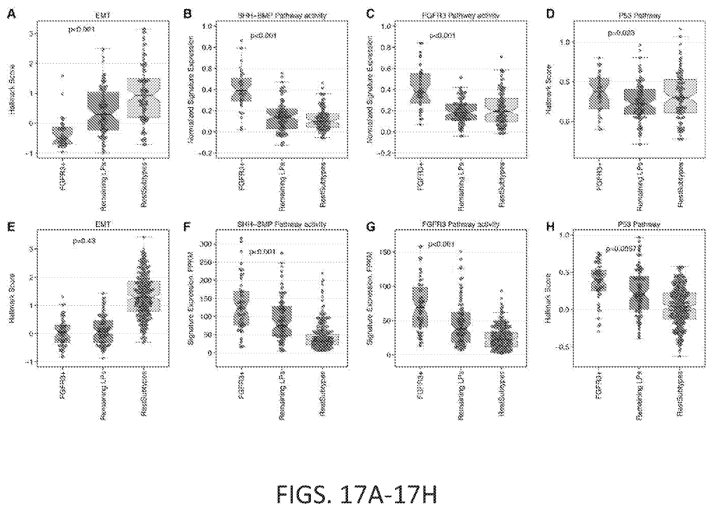

A total of 36/223 (16%) and 55/408 (14%) FGFR3+ cases in the NAC and TCGA cohorts were identified, respectively. The majority of the FGFR3+ calls in both cohorts were of the luminal-papillary mRNA subtype (see Table 8). In both cohorts, patients with FGFR3+ tumors had better survival than other patients (p=0.001 and p=0.003 for NAC and TCGA, respectively) (see FIGS. 16A, 16B). As expected, the FGFR3, SHH and p53 signature scores were significantly higher among FGFR3+ cases when compared to the other tumors. In the NAC cohort, EMT hallmark scores were significantly lower among FGFR3+ cases (p<0.001), whereas FGFR3+ cases from the TCGA cohort showed no significant difference in EMT activity (see FIGS. 17A-17H). FGFR3 was mutated in 25/55 FGFR3+ cases (45%) compared to 32/350 negative cases (9%) from the TCGA cohort (p<0.001). The FGFR3+ cases were depleted for TP53 mutations in 15/55 (27%) compared to 180/350 (51%) negative cases (p<0.001). Likewise, RB1 mutations were fewer in FGFR3+ cases, 0/55 (0%) compared to 70/350 (20%) of negative cases (p<0.001).

TABLE 8
Intersection of the GC FGFR3+ tumors and the TCGA2017
mRNA subtypes for the NAC, TCGA, UTSW and PCC cohorts.

| GC result | FGFR3+ | GC− | Total | FGFR3+ | GC− | Total |
|---|---|---|---|---|---|---|
| | NAC cohort | | | TCGA cohort | | |
| Basal_Sq | 0 | 73 | 73 | 1 | 140 | 141 |
| Luminal | 0 | 1 | 1 | 0 | 25 | 25 |
| Luminal_Inf | 1 | 5 | 6 | 1 | 76 | 77 |
| Lum-pap | 35 | 89 | 124 | 53 | 89 | 142 |
| Neuronal | 0 | 3 | 3 | 0 | 20 | 20 |
| Unknown | 0 | 16 | 15 | na | na | na |
| Total | 36 | 187 | 223 | 55 | 350 | 405 |
| | UTSW cohort | | | PCC cohort | | |
| Basal_Sq | 0 | 33 | 33 | 0 | 79 | 79 |
| Luminal | 0 | 3 | 3 | 3 | 12 | 15 |
| Luminal_Inf | 0 | 8 | 8 | 0 | 24 | 24 |
| Lum-pap | 10 | 26 | 36 | 21 | 74 | 95 |
| Neuronal | 0 | 5 | 5 | 0 | 12 | 12 |
| Unknown | 0 | 9 | 9 | 0 | 30 | 30 |
| Total | 10 | 84 | 94 | 24 | 231 | 255 |

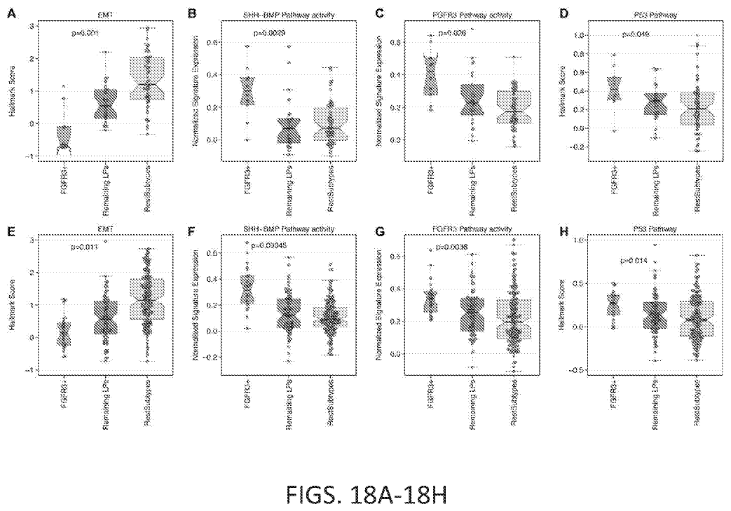

To validate the classifier, an independent RC cohort (UTSW) of 94 patients was used, 10 (11%) FGFR3+ cases (all luminal-papillary) were identified with excellent prognosis (see FIG. 16C) and expected biological character (see FIGS. 18A-18D). Multivariable cox regression analysis revealed that the GC was a significant survival predictor in the NAC TURBT cohort, but not in the TCGA and UTSW cohorts (see Table 9). The GC was also validated in a prospectively collected commercial cohort (PCC, n=225), resulting in 24/225 (11%) FGFR3+ positive cases (21 luminal-papillary, 3 luminal) with genomic characteristics consistent with FGFR3+ cases from the other cohorts (see FIGS. 18A-18H).

TABLE 9
Multivariable analyses in the NAC, TCGA and UTSW cohorts.

| | | | | |
|---|---|---|---|---|
| Variable | Unadjusted HR | p-value | Adjusted HR | p-value |

NAC Cohort

| | | | | |
|---|---|---|---|---|
| Age | 1.02 (1.00-1.05) | 0.027 | 1.03 (1.00-1.05) | 0.037* |
| Gender: Male | 0.97 (0.60-1.56) | 0.9 | 1.12 (0.64-1.95) | 0.7 |
| >ypT2 | 6.59 (4.06-10.70) | p < 0.001 | 5.09 (2.88-8.97) | p < 0.001* |
| pN1-3 | 3.79 (2.31-6.23) | p < 0.001 | 2.45 (1.45-4.15) | p < 0.001* |
| FGFR3+ | 0.14 (0.03-0.57) | 0.006 | 0.17 (0.04-0.70) | 0.014* |

TCGA Cohort

| | | | | |
|---|---|---|---|---|
| Age | 1.04 (1.02-1.05) | p < 0.001* | 1.03 (1.02-1.05) | p < 0.001* |
| Gender: Male | 0.88 (0.64-1.23) | 0.478 | 0.87 (0.61-1.24) | 0.432 |
| >pT2 | 2.08 (1.43-3.02) | p < 0.001* | 1.59 (1.05-2.41) | p < 0.001* |
| pN1-3 | 2.24 (1.63-3.07) | p < 0.001* | 1.85 (1.33-2.58) | 0.028* |
| FGFR3+ | 0.37 (019-0.73) | 0.004* | 0.44 (0.18-1.08) | 0.072 |

UTSW Cohort

| | | | | |
|---|---|---|---|---|
| Age | 1.03 (1.00-1.06) | 0.083 | 1.03 (1.00-1.06) | 0.086 |
| Gender: Male | 0.68 (0.33-1.41) | 0.3 | 0.72 (0.34-1.54) | 0.402 |
| >pT2 | 2.35 (1.23-4.48) | 0.010* | 1.32 (0.62-2.79) | 0.473 |
| pN1-3 | 2.50 (1.38-4.53) | 0.002* | 2.13 (1.08-4.21) | 0.029* |
| FGFR3+ | 0.12 (0.02-0.89) | 0.038* | 0.15 (0.02-1.10) | 0.062 |

### Comparison of the GC Single Sample Classifier to the Consensus Subtyping Model

Finally, the recently released consensus molecular classification of The Bladder Cancer Molecular Taxonomy Group was used to assign tumors from all four cohorts into the six consensus mRNA subtypes (Ba/Sq, LumNS, LumP, LumU, Stroma-rich and NE-like). Intersecting the consensus subtype calls with the results of the GC revealed that the GC identified tumors from all three luminal subtypes (unstable, non-specified or papillary), and only rarely the stromal-rich consensus subtype (see Table 10).

TABLE 10
Intersection of the GC FGFR3+ tumors and the Consensus Classifier
mRNA subtypes for the NAC, TCGA, UTSW and PCC cohorts

| GC result | FGFR3+ | GC− | Total | FGFR3+ | GC− | Total |
|---|---|---|---|---|---|---|
| | NAC Cohort | | | TCGA cohort | | |
| Ba/Sq | 0 | 84 | 84 | 0 | 151 | 151 |
| LumNS | 2 | 17 | 19 | 1 | 20 | 21 |
| LumP | 22 | 25 | 47 | 51 | 77 | 128 |
| LumU | 11 | 45 | 56 | 2 | 51 | 53 |
| NE-Like | 0 | 2 | 2 | 0 | 6 | 6 |
| Stroma-rich | 1 | 14 | 15 | 0 | 45 | 45 |
| Total | 36 | 187 | 223 | 55 | 350 | 405 |
| | UTSW cohort | | | PCC cohort | | |
| Ba/Sq | 0 | 42 | 42 | 0 | 116 | 116 |
| LumNS | 1 | 9 | 10 | 7 | 25 | 32 |
| LumP | 6 | 9 | 15 | 11 | 21 | 32 |
| LumU | 3 | 7 | 10 | 5 | 31 | 36 |
| NE-Like | 0 | 1 | 1 | 0 | 6 | 6 |
| Stroma-rich | 0 | 16 | 16 | 1 | 32 | 33 |
| Total | 10 | 84 | 94 | 24 | 231 | 255 |

These results showed that methods of the present disclosure and lncRNAs are useful for subtyping bladder cancer. These results further showed that the subtyping methods of the present disclosure are useful for prognosing or predicting outcome for a subject with bladder cancer. The results suggested that the subtyping methods of the present disclosure may be used to determine a treatment for a subject with bladder cancer.

What is claimed is:

1. A method comprising:
a) providing a biological sample from a subject having bladder cancer;
b) measuring levels of expression in the biological sample of a plurality of genes selected from Table 2 and/or Table 5; and
c) subtyping the bladder cancer of the subject according to a genomic subtyping classifier based on the levels of expression of the plurality of genes, wherein said subtyping comprises assigning the bladder cancer to one of five subtypes selected from the group consisting of basal/squamous, luminal, luminal-infiltrated, luminal-papillary, and neuronal subtype.

2. The method of claim 1, further comprising determining that the subject has a favorable prognosis if the subtyping indicates that the subject has the luminal-papillary subtype or determining that the subject has an unfavorable prognosis if the subtyping indicates that the subject has the basal/squamous, luminal, luminal-infiltrated, or neuronal subtype.

3. The method of claim 1 or 2, further comprising determining that the subject has a less aggressive tumor if the subtyping indicates that the subject has the luminal-papillary subtype or determining that the subject has a more aggressive tumor if the subtyping indicates that the subject has the basal/squamous, luminal, luminal-infiltrated, or neuronal subtype.

4. The method of any one of claims 1-3, further comprising administering an FGFR3 inhibitor to the subject if the subtyping indicates that the subject has the luminal-papillary subtype and administering neoadjuvant chemotherapy to the subject if the subtyping indicates that the subject has the basal/squamous, luminal, luminal-infiltrated, or neuronal subtype.

5. A method for treating a subject with bladder cancer, the method comprising:
determining the subtype of bladder cancer the subject has by:
obtaining or having obtained a biological sample from the subject;
measuring or having measured the levels of expression in the biological sample of a plurality of genes selected from Table 2 and/or Table 5; and
assigning the bladder cancer to one of five subtypes selected from the group consisting of basal/squamous, luminal, luminal-infiltrated, luminal-papillary, and neuronal subtype based on the levels of expression of the plurality of genes; and
if the subject has luminal-papillary subtype, then administering an FGFR3 inhibitor, and
if the subject has basal/squamous, luminal, luminal-infiltrated, or neuronal subtype, then administering to the subject neoadjuvant chemotherapy.

6. The method of claim 4 or 5, wherein the neoadjuvant chemotherapy comprises administering cisplatin.

7. The method of any one of claims 1-6, wherein the method is performed prior to treatment of the patient with anti-cancer therapy.

8. The method of any one of claims 1-6, wherein the subject is undergoing anti-cancer therapy.

9. The method of any one of claims 1-8, wherein the subject has muscle-invasive bladder cancer.

10. The method of any one of claims 1-9, wherein the biological sample is a biopsy.

11. The method of any one of claims 1-9, wherein the biological sample is a urine sample, a blood sample, or a bladder tumor sample.

12. The method of any one of claims 1-9, wherein the biological sample is a transurethral resection (TUR) specimen.

13. The method of any one of claims 1-12, wherein the subject is a human being.

14. The method of any one of claims 1-13, wherein the level of expression is increased or reduced compared to a control.

15. The method of any one of claims 1-14, wherein said measuring levels of expression comprises performing in situ hybridization, a PCR-based method, a sequencing method, an array-based method, an immunohistochemical method, an RNA assay method, or an immunoassay method.

16. The method of any one of claims 1-15, wherein said measuring levels of expression comprises using a reagent selected from the group consisting of a nucleic acid probe, one or more nucleic acid primers, and an antibody.

17. The method of any one of claims 1-16, wherein said measuring levels of expression comprises measuring the level of an RNA transcript.

18. The method of claim 17, wherein the RNA is long non-coding RNA.

19. The method of any one of claims 1-18, wherein the bladder cancer is FGFR3 positive.

20. The method of any one of claims 1-19, further comprising administering at least one anti-cancer therapy selected from the group consisting of an FGFR3 inhibitor, surgery, radiation therapy, immunotherapy, biological therapy, hormonal therapy, and photodynamic therapy.

21. A method for determining a treatment for a subject who has bladder cancer, the method comprising:
a) providing a biological sample from the subject;
b) detecting the presence or expression level in the biological sample for a plurality of targets selected from Table 2 and/or Table 5;
c) subtyping the bladder cancer of the subject according to a genomic subtyping classifier based on the levels of expression of the plurality of genes, wherein said subtyping comprises assigning the bladder cancer to one of five subtypes selected from the group consisting of basal/squamous, luminal, luminal-infiltrated, luminal-papillary, and neuronal subtype; and
d) determining whether or not the subject is likely to be responsive to anti-cancer therapy based on the subtype of the bladder cancer in the subject; and
e) prescribing anti-cancer therapy to the subject if the patient is identified as likely to be responsive to anti-cancer therapy.

22. The method of claim 21, wherein the anti-cancer therapy is an FGFR3 inhibitor.

23. A kit for prognosing bladder cancer in a subject, the kit comprising agents for detecting the presence or expression levels for a plurality of targets, wherein said plurality of genes comprises one or more genes selected from Table 2 and/or Table 5.

24. The kit of claim 23, wherein said agents comprise reagents for performing in situ hybridization, a PCR-based method, an array-based method, a sequencing method, an immunohistochemical method, an RNA assay method, or an immunoassay method.

25. The kit of claim 23 or 24, wherein said agents comprise one or more of a microarray, a nucleic acid probe, a nucleic acid primer, or an antibody.

26. The kit of any one of claims 23-25, wherein the kit comprises at least one set of PCR primers capable of amplifying a nucleic acid comprising a sequence of a gene selected from Table 2 and/or Table 5 or its complement.

27. The kit of any one of claims 23-26, wherein the kit comprises at least one probe capable of hybridizing to a nucleic acid comprising a sequence of a gene selected from Table 2 and/or Table 5 or its complement.

28. The kit of any one of claims 23-27, further comprising information, in electronic or paper form, comprising instructions on how to determine if a subject is likely to be responsive to anti-cancer therapy.

29. The kit of any one of claims 23-28, further comprising one or more control reference samples.

30. A probe set for prognosing bladder cancer in a subject, the probe set comprising a plurality of probes for detecting a plurality of target nucleic acids, wherein the plurality of target nucleic acids comprises one or more gene sequences, or complements thereof, of genes selected from Table 2 and/or Table 5.

31. The probe set of claim 30, wherein at least one probe is detectably labeled.

32. A kit for prognosing prostate cancer comprising the probe set of claim 30 or 31.

33. A system for analyzing a bladder cancer to provide a prognosis to a subject having bladder cancer, the system comprising:
a) the probe set of claim 30 or 31; and
b) a computer model or algorithm for analyzing an expression level or expression profile of the plurality of target nucleic acids hybridized to the plurality of probes in a biological sample from a subject who has bladder cancer and subtyping the bladder cancer of the subject according to a genomic subtyping classifier based on the expression level or expression profile, wherein said subtyping comprises assigning the bladder cancer to one of five subtypes selected from the group consisting of basal/squamous, luminal, luminal-infiltrated, luminal-papillary, and neuronal subtype.

34. A kit for prognosing prostate cancer in a subject comprising the system of claim 33.

35. The kit of claim 34, further comprising a computer model or algorithm for designating a treatment modality for the subject.

36. The kit of claim 34 or 35, further comprising a computer model or algorithm for normalizing the expression level or expression profile of the plurality of target nucleic acids.

37. A method for treating a subject with bladder cancer, the method comprising: a) providing a biological sample from a subject having bladder cancer; b) detecting the presence or expression level in the biological sample for a plurality of targets selected from Table 2 and/or Table 5; and c) administering a treatment to the subject, wherein the treatment is selected from the group consisting of neoadjuvant chemotherapy or an anti-cancer treatment.

38. The method of claim 37, wherein the anti-cancer treatment is selected from the group consisting of surgery, chemotherapy, radiation therapy, immunotherapy, biological therapy, hormonal therapy, and photodynamic therapy.

39. The method of claim 37 or 38, further comprising subtyping the bladder cancer in the subject according to a genomic subtyping classifier based on the presence or expression levels of the plurality of targets, wherein said subtyping comprises assigning the bladder cancer to one of five subtypes selected from the group consisting of basal/squamous, luminal, luminal-infiltrated, luminal-papillary, and neuronal subtype.
